(19) United States
(12) Patent Application Publication
(10) Pub. No.: US 2023/0381472 A1
(43) Pub. Date: Nov. 30, 2023

(54) Systems and Methods Related to Catheter-Based Procedures

(71) Applicant: Amplitude Vascular Systems, Inc., Boston, MA (US)

(72) Inventors: Robert Chisena, Boston, MA (US); Hitinder Gurm, Boston, MA (US); Louis Cannon, Boston, MA (US)

(21) Appl. No.: 18/199,026

(22) Filed: May 18, 2023

Related U.S. Application Data
(60) Provisional application No. 63/346,704, filed on May 27, 2022.

(51) Int. Cl. A61M 25/10; G06N 5/022

(52) U.S. Cl. CPC A61M 25/104; G06N 5/022; A61M 2205/3303; A61M 2205/3584; A61M 2210/125; A61M 2205/52

(57) ABSTRACT

Methods related to catheter-based procedures are provided. Aspects of the methods of embodiments of the invention include performing a catheter-based procedure and communicating data regarding the catheter-based procedure to and/or from a catheter-based procedure information module. Catheter-based procedure information modules may be remote modules. Also provided are systems comprising a first processor comprising memory operably coupled to the first processor, wherein the memory comprises instructions stored thereon, which, when executed by the first processor, cause the first processor to: receive data regarding a catheter-based procedure from a catheter-based system, and transmit data regarding a catheter-based procedure to a catheter-based system. Also included in systems of embodiments of the invention is a catheter-based system, comprising: a catheter-based device, a second processor comprising memory operably coupled to the second processor, wherein the memory comprises instructions stored thereon, which, when executed by the second processor, cause the second processor to: transmit data regarding the catheter-based procedure to the catheter-based procedure information module, receive data regarding the catheter-based procedure from the catheter-based procedure information module, and configure the catheter-based device based at least in part on data regarding the catheter-based procedure received from the catheter-based procedure information module. Also included in systems of embodiments of the invention is an operable connection between the catheter-based procedure information module and the catheter-based system. In addition, non-transitory computer readable storage mediums configured to perform the methods described herein are provided. The methods, systems and non-transitory computer readable storage mediums find use in a variety of different applications, including balloon angioplasty applications or other catheter-based procedures, therapies or treatments.

### CROSS-REFERENCE TO RELATED APPLICATIONS

Pursuant to 35 U.S.C. § 119(e), this application claims priority to the filing date of U.S. provisional patent application Ser. No. 63/346,704 filed May 27, 2022, the disclosures of which application is incorporated herein by reference in its entirety.

### INTRODUCTION

Cardiovascular tissue is susceptible to atherosclerotic plaque build-up through a mechanism called atherosclerosis, which is the accumulation of fatty and calcified materials that cause stenosis, the narrowing of an arterial lumen, or the failure of heart valves to function properly. Catheter-based procedures, for example, deploying catheter-based systems, such as angioplasty balloons via a catheter, are an important technique for addressing such disease conditions in cardiovascular tissue or in other tissues affected by disease conditions that cause the narrowing of luminal tissues. Certain catheter-based procedures involve deploying a catheter-based system configured to apply pulsatile energy, e.g., via a balloon, to tissue, such as cardiovascular tissue. Such procedures and systems may be configurable based on several different parameters, including a frequency of pulsatile energy applied to tissue, an amount of pressure applied by pulsatile energy to tissue, the number of pulses of pulsatile energy applied to tissue, a duty cycle of applying pulsatile energy to tissue, a duration of treatment, increases or decreases in frequency or pressure, rates of such increases or decreases, synchronizing different aspects of treatment with physiological conditions, e.g., a cardiac cycle, or interleaving applying pulsatile energy with applying static pressure to tissue, for example. Existing treatments involving such catheter-based procedures and systems may rely exclusively on operator determinations or judgment in connection with configuring such procedures or systems and as such may lack the ability to readily and fully take into account past performance and past learning from similar or analogous catheter-based procedures, e.g., performed at different treatment centers and/or on different subjects.

### SUMMARY

Therefore, there remains a need for improved configuration and control of catheter-based treatment procedures and methods of performing catheter-based procedures, including in connection with initiating treatment (i.e., automatically generating initial configuration settings) or dynamically adjusting aspects of treatment (i.e., automatically generating revised configuration settings for adjusting settings on the fly during treatment based on feedback obtained during treatment in real time or nearly in real time). For example, methods and systems for identifying, and presenting for operator approval and/or amendment (i.e., enabling an operator of a catheter-based system to accept an automatically generated configuration as presented, or, alternatively, to adjust one or more aspects of an automatically generated configuration prior to applying such configuration settings), configuration settings for catheter-based procedures could improve the effectiveness of such procedures, including for example, among a plurality of different subjects at a plurality of different treatment locations. In addition, such methods and systems could improve the safety of performing catheter-based procedures, e.g., by terminating a catheter-based procedure prior to device or tissue failure.

Methods and systems of the present invention may work to identify configuration settings for catheter-based procedures by leveraging data regarding historical catheter-based procedures such that subsequent catheter-based procedures are informed based on such past procedures. Methods and systems of the present invention may be configured to provide a centralized repository of such procedure-based data so that catheter-based procedures that occur at a plurality of diverse locations on a plurality of diverse subjects may gain access to the same broad data set of historical results of catheter-based procedures. Methods and systems of the present invention may directly configure catheter-based procedures or systems based on automatically generated configuration settings or may interface with an operator of a catheter-based system by, for example, proposing a suggested initial or revised configuration of a catheter-based procedure or system for approval or amendment by an operator prior to applying to the catheter-based procedure or catheter-based system.

Aspects of the methods of embodiments of the present invention include performing a catheter-based procedure and communicating data regarding the catheter-based procedure to and/or from a catheter-based procedure information module. Catheter-based procedure information modules may be remote modules. Also provided are systems comprising a first processor comprising memory operably coupled to the first processor, wherein the memory comprises instructions stored thereon, which, when executed by the first processor, cause the first processor to: receive data regarding a catheter-based procedure from a catheter-based system, and transmit data regarding a catheter-based procedure to a catheter-based system. Also included in systems of embodiments of the invention is a catheter-based system, comprising: a catheter-based device, a second processor comprising memory operably coupled to the second processor, wherein the memory comprises instructions stored thereon, which, when executed by the second processor, cause the second processor to: transmit data regarding the catheter-based procedure to the catheter-based procedure information module, receive data regarding the catheter-based procedure from the catheter-based procedure information module, and configure the catheter-based device based at least in part on data regarding the catheter-based procedure received from the catheter-based procedure information module. Also included in systems of embodiments of the invention is an operable connection between the catheter-based procedure information module and the catheter-based system. In addition, non-transitory computer readable storage media configured to perform the methods described herein are provided. The methods, systems and non-transitory computer readable storage media find use in a variety of different applications, including balloon angioplasty applications or other catheter-based procedures, therapies or treatments.

### BRIEF DESCRIPTION OF THE FIGURES

FIG. 1 provides a schematic diagram of a catheter-based system for imparting pulsatile energy to cardiovascular tissue for use in connection with embodiments of methods and systems of the present invention.

FIG. 2 provides a flow diagram illustrating a method related to a catheter-based procedure according to an embodiment of the invention.

FIG. 3 provides a schematic diagram of a control system of a catheter-based system and the interface with an output, comprising a treatment plan, of a catheter-based procedure information module.

FIG. 4 depicts a schematic of a robotic method for delivering therapy energy to a diseased tissue from a control room.

FIG. 5 depicts pressure-volume curves of tissue before and after a catheter-based procedure.

FIGS. 6A and 6B provide an example of measurements of changes in tissue compliance obtained during a catheter-based procedure.

FIG. 7 provides two examples of experimentally measured pressure in a balloon used in a catheter-based procedure and force output from such balloon.

FIG. 8 is a graph illustrating that as frequency (or pressure difference) of oscillations of a balloon used in a catheter-based procedure changes, the catheter-based procedure is controlled to ensure the balloon is evacuated to within a desired pressure range according to the principles of the present teachings.

FIG. 9 presents exemplary images of calcifications in luminal tissue prior to and subsequent to applying a catheter-based procedure of the present invention.

FIG. 10 presents a table summarizing the expected effectiveness of certain catheter-based procedures under differing circumstances.

FIG. 11 provides an X-ray image of a calcium deposit.

### DETAILED DESCRIPTION

Methods related to catheter-based procedures are provided. Aspects of the methods of embodiments of the invention include performing a catheter-based procedure and communicating data regarding the catheter-based procedure to and/or from a catheter-based procedure information module. Catheter-based procedure information modules may be remote modules. Also provided are systems comprising a first processor comprising memory operably coupled to the first processor, wherein the memory comprises instructions stored thereon, which, when executed by the first processor, cause the first processor to: receive data regarding a catheter-based procedure from a catheter-based system, and transmit data regarding a catheter-based procedure to a catheter-based system. Also included in systems of embodiments of the invention is a catheter-based system, comprising: a catheter-based device, a second processor comprising memory operably coupled to the second processor, wherein the memory comprises instructions stored thereon, which, when executed by the second processor, cause the second processor to: transmit data regarding the catheter-based procedure to the catheter-based procedure information module, receive data regarding the catheter-based procedure from the catheter-based procedure information module, and configure the catheter-based device based at least in part on data regarding the catheter-based procedure received from the catheter-based procedure information module. Also included in systems of embodiments of the invention is an operable connection between the catheter-based procedure information module and the catheter-based system. In addition, non-transitory computer readable storage media configured to perform the methods described herein are provided. The methods, systems and non-transitory computer readable storage media find use in a variety of different applications, including balloon angioplasty applications or other catheter-based procedures, therapies or treatments.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, embodiments of the methods are described first in greater detail, followed by a review of embodiments of the systems and non-transitory computer readable storage media for practicing the subject methods.

### Methods

As summarized above, methods related to catheter-based procedures are provided. Embodiments of methods of the invention comprise performing a catheter-based procedure and communicating data regarding the catheter-based procedure to and/or from a catheter-based procedure information module. In embodiments, the catheter-based procedure information module is a remote module. For example, the catheter-based procedure information module may be located in a different room, city or country from where the catheter-based procedure is performed. In other embodiments, data regarding the catheter-based procedure communicated to and/or from the catheter-based procedure information module comprises one or more of pre-treatment data, peri-treatment data or post-treatment data, as described below.

Methods of the invention find use in a variety of applications, including angioplasty applications or other catheter-based therapies or treatments, e.g., treatments of heart valve disease. In some instances, the methods find use in fracturing hardened materials, e.g., calcium deposits, embedded within cardiovascular tissue, e.g., a heart valve leaflet or heart valve commissure or other cardiovascular tissue. Specifically, methods of embodiments of the invention find use in conducting catheter-based procedures including, for example, leveraging the results of previously performed catheter-based procedures to better inform configurations applied when performing subsequent catheter-based procedures, including on the same or different subjects at the same or different treatment locations. The present disclosure describes applications of embodiments related to treating tissue, such as cardiovascular tissue, related to, e.g., calcifications and/or depositions within tissue, such as cardiovascular tissue, such as heart valves or features thereof or tissue supporting heart valves. However, the present system and teachings are not solely limited to catheter-based procedures for addressing cardiovascular tissue in connection with calcifications and may be generally applied to other applications as determined by those skilled in the art.

The methods may be used in connection with performing catheter-based procedures on any number of different subjects. In some instances, the subjects are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some instances, the subjects are humans.

### Catheter-Based Procedure Information Module:

Embodiments of methods of the present invention include communicating data regarding a catheter-based procedure to and/or from a catheter-based procedure information module. As such, in embodiments, the catheter-based procedure information module may function in a manner similar to a controller, receiving data regarding the catheter-based procedure and/or transmitting configurations or settings for performing the catheter-based procedure, such as configurations or settings selected to automate the catheter-based procedure or to improve the effectiveness of the catheter-based procedure.

In embodiments, the catheter-based information module may comprise a processor with memory configured to communicate data regarding the catheter-based procedure. While no specific processor (or other computer hardware) is required to practice the present invention, nonetheless processors of interest may include general purpose processors, graphics processing units, systems on a chip, micro-controllers or the like, such as those that are commercially available from AMD, ARM, Broadcom, Intel, Motorola, Nvidia, Qualcomm, Texas Instruments or the like. In some cases, the catheter-based information module may comprise more than one processor interconnected via an interconnection network or the like. Memory may be any suitable device in which the processor can store and retrieve data, such as magnetic, optical, or solid-state storage devices (including magnetic or optical disks or tape or RAM, or any other suitable device, either fixed or portable).

In embodiments, any convenient means of communicating to and/or from the catheter-based procedure information module may be employed. In some cases, the catheter-based procedure information module is operably connected to a catheter-based system for performing the catheter-based procedure. That is, the catheter-based procedure information module may be directly or indirectly coupled to a catheter-based system for performing the catheter-based procedure. In other cases, the catheter-based procedure information module and/or a catheter-based system for performing the catheter-based procedure may be configured to include any convenient communication interface. In some embodiments, the communication interface includes a receiver and/or transmitter for communicating with a network and/or another device.

A communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols and cellular communication, such as code division multiple access (CDMA) or Global System for Mobile communications (GSM)). In some cases, an I2C communication protocol, i.e., an inter-integrated circuit communication protocol, may be employed by the communication interface, e.g., over a bus, such as a serial bus, thereof. In some cases, communication interfaces of interest may be communication interfaces designed to reduce or minimize wire count, such as wire count between integrated circuits or a wire count of a catheter-based system for performing a catheter-based procedure.

In one embodiment, a communication interface is configured to include one or more communication ports, e.g., physical ports or interfaces such as a USB port, an RS-232 port, or any other suitable electrical connection port to allow data communication to and/or from the catheter-based procedure information module and other external devices such as a catheter-based system for performing the catheter-based procedure or a computer terminal (for example, at a control room) that is configured for similar complementary data communication.

In one embodiment, the communication interface is configured for infrared communication, Bluetooth® communication, or any other suitable wireless communication protocol to enable the catheter-based procedure information module to communicate with other devices such as a catheter-based system for performing the catheter-based procedure, computer terminals and/or networks, communication enabled mobile telephones, personal digital assistants, or any other communication devices which may be used in conjunction with the catheter-based procedure information module.

In one embodiment, the communication interface is configured to provide a connection for data transfer utilizing Internet Protocol (IP) through a cell phone network, Short Message Service (SMS), wireless connection to a personal computer (PC) on a Local Area Network (LAN), which is connected to the internet, or WiFi connection to the internet at a WiFi hotspot.

In one embodiment, the catheter-based procedure information module is configured to wirelessly communicate with a server device via a communication interface, e.g., using a common standard such as 802.11 or Bluetooth® RF protocol or an IrDA infrared protocol. Such a server device may be a portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touchscreen.

In some embodiments, the communication interface is configured to automatically or semi-automatically communicate data stored in the catheter-based procedure information module, e.g., in an optional data storage unit, with a network or server device using one or more of the communication protocols and/or mechanisms described above.

In embodiments, the catheter-based procedure information module is a remote module. By “remote module,” it is meant that the catheter-based procedure information module is removed at some distance from the location where the catheter-based procedure is conducted. That is, the catheter-based information module may be separate from the catheter device involved in performing the catheter-based procedure, i.e., separate physical and/or logical devices. For example, the catheter-based procedure information module may be a separate module from the catheter device (catheter-based system) used in connection with performing the catheter-based procedure, and the catheter-based procedure information module may be located in a different part of, e.g., a different partition of, a treatment or procedure room, such as beyond a sterile field or behind a barrier, such as a curtain or a lead shield or the like, where the catheter-based procedure is performed. In other examples, the catheter-based procedure information module may be located in a different room of the same building, such as for example, a control room of a hospital or treatment center. In other cases, the catheter-based procedure information module may be located in a different building or city or country from where the catheter-based procedure is performed, i.e., at any convenient distance, e.g., as called for by the constraints and/or configurations of the catheter-based procedure information module and the catheter-based procedure.

In embodiments, the catheter-based procedure information module is configured to store data regarding the catheter-based procedure. The catheter-based procedure information module may be configured to store any data pertinent to the catheter-based procedure, such as, for example, pre-treatment data, peri-treatment data and post-treatment data, as described herein. Such data may be stored in a manner or format that enables the data to be readily accessible, e.g., for analysis or as input to an algorithm, such as an artificial intelligence algorithm, e.g., a machine learning algorithm or neural network algorithm or deep learning algorithm or convolutional neural network algorithm, configured to generate settings and/or configurations for a catheter-based procedure, as described herein. Such data may be stored in a manner or format that facilitates such analysis, such as in a database format or other data structure format such that data regarding different catheter-based procedures is stored in a uniform and/or predictable manner capable of ready access as needed. For example, data regarding the catheter-based procedure may be stored in a manner that enables such data to be accessed by more than one catheter-based procedure information module, e.g., different catheter-based procedure information modules present at different control rooms of different treatment centers, such as, for example, stored in distributed storage or cloud-based storage or the like.

In some embodiments, a memory of the catheter-based procedure information module may be configured to store such data and/or the catheter-based procedure information module may be configured to store such data in a separate external storage, including, for example, in an external storage attached via a network connection, such as distributed data storage or cloud-based storage, i.e., where data is stored at more than one node of a computer network and/or in a logically contiguous manner distributed over physically separated storage devices including at separate locations interconnected, e.g., via a computer network. Any convenient distributed data storage and/or cloud-based storage techniques may be applied, including for example, commercially available cloud-based storage, such as cloud storage services available from Amazon, Box, Dropbox, Google or Microsoft, for example. In embodiments, data storage, including network-based external storage and/or cloud-based storage, may be configured to be compliant with applicable security and privacy-related laws, regulations, rules, standards and best practices, such as, for example, the Health Insurance Portability and Accountability Act of 1996 (HIPPA) in connection with storing medical data in the United States.

In embodiments, the catheter-based procedure information module may be configured to receive and/or transmit data regarding a plurality of catheter-based procedures. In some cases, the catheter-based procedure information module may be configured to receive and/or transmit data regarding a first catheter-based procedure at a first time and a second catheter-based procedure at a second time. As such, the catheter-based procedure information module may be configured to receive and/or transmit data regarding catheter-based procedures that occur over any desired span of time, such as procedures that occur over the course of a day or more, such as over the course of a month or more or over the course of a year or more.

In other cases, the catheter-based procedure information module may be operably connected, e.g., via a computer network or the like, such as described above, to more than one catheter-based system, i.e., systems for performing catheter-based procedures. In such cases, the catheter-based procedure information module may receive and/or transmit data regarding more than one catheter-based treatment substantially simultaneously. In embodiments, the different catheter-based procedures are performed at different locations, i.e., different treatment locations, such as at different treatment rooms or procedure rooms of a treatment center or hospital or in different buildings such as different treatment centers or different hospitals or in different cities or different countries.

In embodiments, the catheter-based procedure information module is configured to receive, store and/or analyze data regarding any convenient number of catheter-based procedures (i.e., data originating from any number of catheter-based procedures) as desired, such as one or more, such as ten or more, such as 100 or more, such 1,000 or more, such as 10,000 or more, such as 100,000 or more catheter-based procedures.

### Catheter-Based Procedure Data:

In embodiments, data regarding the catheter-based procedure communicated to and/or from the catheter-based procedure information module comprises any available data relevant to the catheter-based procedure and may vary as desired. In some cases, data regarding the catheter-based procedure is data relevant for the purpose of, for example, generating configuration settings for, and/or analyzing the effectiveness of, the catheter-based procedure or other, e.g., subsequently performed, catheter-based procedures, including subsequent catheter-based procedures performed on different subjects.

In some embodiments, data regarding the catheter-based procedure communicated to and/or from the catheter-based procedure information module comprises pre-treatment data or peri-treatment data or post-treatment data. “Pre-treatment data” refers to information that is available prior to performing the catheter-based procedure; “peri-treatment information” refers to information that is available during the catheter-based procedure, i.e., data that is available while performing the catheter-based procedure; and “post-treatment data” refers to information that is available after performing the catheter-based procedure. As such, in embodiments, pre-treatment data communicated to and/or from the catheter-based procedure information module comprises data collected prior to performing the catheter-based procedure; peri-treatment data communicated to and/or from the catheter-based procedure information module comprises data collected during the catheter-based procedure; and post-treatment data communicated to and/or from the catheter-based procedure information module comprises data collected after performing the catheter-based procedure.

Data regarding the catheter-based procedure communicated to and/or from the catheter-based procedure information module may comprise imaging data, such as pre-treatment imaging, peri-treatment imaging and/or post-treatment imaging. Imaging data, such as pre-treatment imaging, peri-treatment imaging and/or post-treatment imaging may comprise images showing one or more characteristics of tissue on which the catheter-based procedure is performed. Such characteristics may comprise characteristics of diseased tissue, e.g., the presence, location or extent of calcium deposits in cardiovascular tissue, such as calcifications of heart valve commissures or leaflets, or other atherosclerotic tissue, such as obstructions in luminal tissue, such as a stenotic artery, including chronic total occlusions. In embodiments, imaging data, such as pre-treatment imaging, peri-treatment imaging and/or post-treatment imaging comprises results of any convenient imaging technology and such may vary, such as, for example, angiography imaging, computed tomography imaging, optical coherence tomography, intravascular ultrasound imaging, echocardiogram imaging or the like. In other embodiments, imaging data, comprises images of aspects of a system for performing a catheter-based treatment, such as images of a catheter balloon present in luminal tissue or a heart-tissue-conforming element present in a heart valve, as such is described in U.S. Application Ser. No. 63/346,703 titled “Systems and Methods for Treating Cardiovascular Tissue” and filed on event date herewith (Attorney Docket No. AVSI-004PRV); the disclosure of which is incorporated herein by reference. In some cases, imaging data comprises annotated images of tissue, such as annotations indicating the location of, or the extent of, diseased tissue, and/or of annotated images of components used in connection with the catheter-based procedure, such as, for example, images of a balloon attached to a catheter present near cardiovascular tissue, e.g., a heart valve with calcium deposits on the leaflets.

Data regarding the catheter-based procedure communicated to and/or from the catheter-based procedure information module may also comprise a vital sign, such as a pre-treatment vital sign, a peri-treatment vital sign or a post-treatment vital sign. A vital sign, such as a pre-treatment vital sign, a peri-treatment vital sign or a post-treatment vital sign, may comprise any available physiological characteristic of a subject and such may vary, for example, depending on the nature of the diseased tissue, underlying condition of a subject or catheter-based procedure. Vital signs of interest include a body temperature, a pulse rate and/or a respiration rate. Vital signs of interest may also comprise other physiological characteristics, such as, a blood pressure measurement, including a systolic and/or diastolic measurement, a blood oxygenation measurement (i.e., a pulse oximeter reading), height or weight measurements, body composition information, other measurements, such as a distance between a catheter entry point (e.g., via a femoral artery) and a treatment site (e.g., an aortic heart valve) or the like.

Data regarding the catheter-based procedure communicated to and/or from the catheter-based procedure information module may also comprise other measurements, such as pre-treatment measurements, peri-treatment measurements and/or post-treatment measurements. For example, such measurements may comprise a ventricular pressure, an aortic pressure, results of an electrocardiogram or blood volume measurements, or measurements of blood perfusion, e.g., perfusion through luminal tissue past an obstruction such as an arterial stenosis.

Data regarding the catheter-based procedure communicated to and/or from the catheter-based procedure information module may also comprise catheter system sensor data. Cather system sensor data may comprise data collected via an aspect of a catheter system used in connection with performing the catheter-based procedure. In embodiments, catheter system sensor data may comprise data collected from sensors attached to or integrated with such a catheter system. In some cases, catheter system sensor data may comprise a pressure measurement, such as a catheter or balloon pressure, or a volume measurement, such as a change in fluid volume. For example, catheter system sensor data may comprise a balloon location, a balloon deployment percentage, a valve opening amount, an eccentricity of the valve, a paravalvular leakage, a surrounding pressure on the valve, a proximal pressure, a distal pressure or a position of the valve in a valve annulus.

Data regarding the catheter-based procedure communicated to and/or from the catheter-based procedure information module may also comprise outcome information. Outcome information may refer to an outcome of the catheter-based procedure and may comprise any aspect of an outcome or result, such as resulting changes in a tissue characteristic, e.g., a post-treatment compliance measurement or a post-treatment measure of heart valve function, in each case upon completion of the catheter-based procedure. In embodiments, outcome information comprises data regarding acute valve opening, valvular compliance (or other compliance metrics, e.g., vascular compliance, based on measurements of pressure and volume changes), intravascular pressure or external pressure. In some cases, relevant pressure data comprises, e.g., blood pressure measurements via a blood pressure cuff for measuring systole and diastole pressures or a ratio of blood pressure measurements obtained via blood pressure cuffs from a leg region (e.g., an ankle) and an arm region of a subject. In other cases, relevant pressure data comprises pressure measurements obtained within the coronary arteries and the aorta or the left ventricle, including, for example, appropriate ratios of various pressures such as diastolic gradient, wave free instantaneous diastolic gradient or a hyperemic fractional flow reserve. Outcome information may comprise data collected from aspects of a catheter system used to perform the catheter-based procedure, such as, for example, pressure or volume sensors integrated into the catheter system, e.g., a volume sensor integrated into a connector of the catheter system (such as a Hall sensor and magnet configured to measure changes in fluid volume). In other cases, outcome information may comprise data collected from sensors or the like that are separate from and external to the catheter system used to perform the catheter-based procedure, such as, for example, imaging results or electrocardiogram results or echocardiogram results or measures of fluid perfusion in certain tissue areas or the like. In embodiments, such sensors may be invasive or non-invasive. In some cases, outcome information may comprise data collected from a mechanism configured to measure pressure across a lesion, such as, for example, measurements obtained via a wire attached to a distal region of a catheter configured to measure proximal and distal pressure across a lesion. In other cases, outcome information may comprise fractional flow reserve (FFR) data. In an embodiment configured to treat aortic valves, outcome information may be obtained using a pressure sensor configured to measure pressure across a treatment zone (e.g., a catheter balloon present in a vessel). In embodiments, a sensor used to obtain outcome information may comprise a fiber optic wire attached to a distal region of a catheter. In still other embodiments, outcome information may comprise data collected via an ultrasonic or magnetic resonance imaging (MRI) or optical imaging system configured to show the position of cardiac structures or changes in cardiac function or flow.

Data regarding the catheter-based procedure that is pre-treatment data, such as pre-treatment imaging or a pre-treatment vital sign or a pre-treatment measurement may comprise data available at any time prior to performing the catheter-based procedure, such as, for example, pre-treatment data collected as part of a pre-procedure work-up of a subject that occurs, for example, the day of the catheter-based procedure or substantially immediately prior to performing the catheter-based procedure or data obtained one or more day before performing the catheter-based procedure, e.g., results of an echocardiogram obtained at an earlier time at a separate, specialized treatment site.

Data regarding the catheter-based procedure that is peri-treatment data, such as peri-treatment imaging or a peri-treatment vital sign or a peri-treatment measurement comprises data available at any time during the catheter-based procedure, such as, for example, data collected substantially at the time the catheter-based procedure begins, e.g., when treatment is first applied via the catheter-based procedure, or at later time when treatment is continued via the catheter-based procedure. Examples of peri-treatment data include imaging data collected at regular intervals through the catheter-based procedure that depict the location of or orientation of an aspect of a catheter-based system used for the catheter-based procedure, or measurements of tissue compliance, i.e., indicating a degree of disruption of calcium deposits, collected at regular intervals, or upon the occurrence of specified events (e.g., changes in pressure or volume greater than a specified threshold), throughout the catheter-based procedure.

Data regarding the catheter-based procedure that is post-treatment data, such as post-treatment imaging or a post-treatment vital sign or a post-treatment measurement may comprise data available at any time after the completion of the catheter-based procedure, such as, for example, data collected substantially immediately after the catheter-based procedure ends, e.g., just after treatment is no longer applied via the catheter-based procedure, or at a later time, such one minute or more, one hour or more, one day or more, one week or more after the catheter-based procedure is completed. Examples of post-treatment data include imaging data collected at regular intervals that depict the location of or orientation of an aspect of a catheter-based system used for the catheter-based procedure, or measurements of tissue compliance, i.e., indicating a degree of disruption of calcium deposits, or other measurements intended to measure an aspect of a subject's physiology affected by the disease condition, in each case collected at regular intervals throughout the catheter-based procedure.

### Configurations for Catheter-Based Procedures:

Methods according to the present invention may further comprise configuring the catheter-based procedure based at least in part on data communicated to and/or from the catheter-based procedure information module. For example, the catheter-based procedure information module may communicate, i.e., transmit, settings to a catheter system used in connection with performing the catheter-based procedure where the settings reflect a specified configuration of the catheter system for applying the catheter-based procedure in a specified manner. Any potential setting or configuration may be transmitted from the catheter-based procedure information module, including, but not limited to, a catheter balloon pressure, such as a maximum pressure, balloon inflation frequencies or duty cycles, total treatment time, total number of balloon pulses, specifications for variations, i.e., changes, in any configurations over time (e.g., a configuration that a catheter balloon pressure will increase at a specified rate over time prior to decreasing at a specified rate over time), or the like. In other cases, potential settings or configurations comprise settings related to hardware type, such as what type (e.g., size or other feature or characteristic) of a catheter balloon or distal crosser unit or heart-tissue-conforming element or catheter or other aspect of a catheter-based system for use in performing the catheter-based procedure should be applied when performing the catheter-based procedure. That is, configurations or settings communicated from the catheter-based procedure information module may comprise at least specifications for aspects of a catheter system used to perform the catheter-based procedure as well as settings for how to operate or control or manipulate such catheter system in connection with performing the catheter-based procedure.

In embodiments, by “configuring the catheter-based procedure based at least in part on data communicated to and/or from the catheter-based procedure information module,” it is meant that the catheter-based information module is configured to first receive information regarding a catheter-based procedure, and, based on such information, communicate information regarding configuring the catheter-based procedure. For example, the catheter-based procedure information module may receive information indicating that a catheter-based procedure is performed to address a diseased heart valve with certain tissue characteristics, and the catheter-based information module may transmit a configuration (i.e., configuration settings) comprising applying pulsatile energy to the diseased tissue at a specified frequency and/or duty cycle and/or duration (e.g., time or total number of pulses) such that the catheter-based procedure is configured based at least in part on such configuration generated by and received from the catheter-based information module. Continuing such example, the catheter-based procedure information module may subsequently receive information indicating a change in tissue compliance (e.g., heart valve compliance), and the catheter-based information module may transmit further, i.e., a revised or updated, configuration settings comprising applying pulsatile energy to the diseased tissue at a different specified frequency and/or pressure and/or duty cycle and/or duration (e.g., time or total number of pulses) or other setting such that the catheter-based procedure is re-configured based at least in part on such subsequent configuration from the catheter-based information module. In some cases, information regarding perfusion past an aspect of a catheter-based system used to perform the catheter-based procedure (e.g., an angioplasty balloon or heart-tissue-conforming element or the like) may be communicated to and/or from a catheter-based information module, such that a revised treatment plan relates to improving perfusion past such aspect of the catheter-based system, e.g., to prevent tissue damage resulting from reduced perfusion past the catheter-based system.

The catheter-based information module of embodiments of the present invention may be configured to generate a treatment plan. In embodiments, the catheter-based procedure information module is configured to implement a treatment plan generation algorithm. In some cases, the treatment plan generation algorithm is configured to generate a treatment plan based at least in part on data regarding the catheter-based procedure communicated to the catheter-based procedure information module. In other cases, data communicated from the catheter-based procedure information module comprises a treatment plan.

By “treatment plan,” it is meant any potential configuration related to performing a catheter-based procedure, i.e., a setting for any configurable aspect of performing a catheter-based procedure. In embodiments, the treatment plan comprises one or more configurations, i.e., settings, for a catheter system for performing the catheter-based procedure. For example, a treatment plan may specify a device type, such as a type of catheter device, such as a catheter device capable of delivering pulsatile energy, e.g., a device capable of delivering high-volume, low-frequency and low-pressure pulses or a device capable of delivering low volume, high-frequency and high-pressure pulses. In other examples, a treatment plan may specify a balloon type, such as, for example, a catheter balloon's size or shape or an expansion volume or degree of compliance, or analogous aspects of other relevant devices, such as a type of heart-tissue-conforming element (e.g., an aspect of a catheter system configured to delivery pulsatile energy to heart valves, such as heart valve commissures or leaflets) or a distal crosser unit (e.g., an aspect of a catheter system configured to traverse chronic total occlusions). In other cases, a treatment plan may specify other aspects of a catheter system for performing a catheter-based procedure, such as specifying a potential source, e.g., a potential source comprising pressurized carbon dioxide, or specifying an oscillator type, e.g., an oscillator configured to generate pressure oscillations at a specified frequency, or specifying a connector type, e.g., a connector configured to transduce a first pulse energy into a second pulse energy with a specified volume of fluidic displacement.

In other embodiments, the treatment plan comprises a configuration of the catheter-based procedure. That is, in some cases, the treatment plan may specify how a catheter system is used to perform a catheter-based procedure. In some cases, the configuration of the catheter-based procedure comprises a treatment type, a treatment duration, a treatment intensity or a treatment frequency. Treatment types may include any desired catheter-based procedure, such as, for example, application of pulsatile energy using a balloon catheter system or application of pulsatile energy to a heart valve using a heart-tissue conforming element. Other examples of treatment types include the application of static energy, e.g., static pressure, to tissue or any desired combination of static and dynamic energy, e.g., a combination of static pressure and pulsatile pressure. Still other examples of treatment types include other treatments performed using catheter-based procedures as such are known in the art, including, e.g., application of energy to tissue in the form of an electrical impulse such that a cavitation bubble, ultrasound wave, plasma bubble or other high-pressure impulse, or in the form of an optical or laser pulse such that a cavitation bubble, ultrasound wave, plasma bubble or other high-pressure impulse is generated, or in the form of heat energy or energy, for example, for cryotherapy, i.e., applying heat or cold to tissue. Treatment intensities may include any desired characteristic of how the treatment type is applied, such as, for example, a pressure applied via pulsatile energy or a duration, such as a total time, during which pulsatile energy is applied. Treatment frequency may include a pulse frequency or duty cycle at which pulsatile energy is applied.

In some cases, treatment plans may relate to (i.e., may be based at least in part on and/or may provide guidance, such as configuration settings) aspects of a device deployed or implanted in tissue, such as a stent. Treatment plan information relate to the deployment of a stent in relevant tissue, the behavior of a stent present in relevant tissue, the response of a stent in relevant tissue, the response of relevant tissue to the placement of a stent and/or the orientation of a stent in relevant tissue. In other embodiments, catheter-based procedures may comprise installing a replacement heart valve, such as an artificial heart valve or a donor heart valve. In such embodiments, a treatment plan may comprise guidance intended to influence and/or predict the desired behavior of the device, such as an implanted device. For example, in the case of a catheter-based procedure for deploying a stent, treatment plan information may relate to predictions regarding how such stent will respond when deployed, e.g., will it jump forward when using a self-expanding stent or how will the stent and vessel tortuosity respond when a stent is deployed or will the stent cause an edge dissection or will the stent fully expand or does the vessel need more preparation. In the case of a catheter-based procedure for deploying a replacement heart valve, treatment plan information may relate to predictions regarding how the replacement heart valve will seat itself and function.

In some cases, a goal of a catheter-based procedure, and therefore a corresponding treatment plan for such catheter-based procedure, is to apply a certain peak pulsatile force to tissue surrounding, for example, a balloon or heart-tissue-conforming element involved in performing a catheter-based procedure. Such pulsatile force may change based on, for example, the stenosis location and the radial calcium location within the tissue. The smaller the diameter of the tissue, the larger the force and the lower the pressure/energy that would need to be applied for the same effect. For calcium that is located further from the center of tissue undergoing treatment, e.g., a lumen, higher pressures are required to achieve the same forces and stresses in the calcium. In embodiments, it is desirable to measure this diameter in situ, either through volume/diameter measurements (i.e., through the balloon), through optical coherence tomography (OCT) or through other imaging techniques, such as ultrasound imaging or fluoroscopy imaging or other external imaging techniques. Once measured, a treatment plan may be developed based at least in part on such measurements such that a peak pressure is applied and then adjusted over the course of performing the catheter-based procedure such that the force/energy applied to the calcium through, for example, a balloon or heart-tissue conforming element, is always consistent. Such examples illustrate how embodiments of the present invention may be used to improve treatment safety, i.e., to improve the safety of performing a catheter-based procedure. For example, embodiments of the present invention comprise providing guidance and/or deciding and/or controlling when to cease treatment. In some embodiments, a catheter-based procedure information module or a controller of a catheter-based system monitors a catheter-based procedure and may cause the termination of such procedure. For example, embodiments may cause a catheter-based procedure to be stopped in the event that a catheter-based procedure information module or a controller of a catheter-based system determines that a balloon of a catheter-based system is likely to rupture. In some cases, embodiments are configured to reduce pressure, e.g., pressure applied to a balloon or heart-tissue conforming element of catheter-based procedure prior to calcium fracturing so as to reduce dissection or reduce energy of a pulse crosser to minimize tissue damage when not in contact with calcium.

In embodiments, the treatment plan generation algorithm may be configured to generate a treatment plan, i.e., configuration settings for performing a catheter-based procedure, in any convenient manner and such may vary. For example, the treatment plan generation algorithm may comprise a look-up algorithm, e.g., analogous to accessing a database, e.g., querying a database based on any relevant aspect of the catheter-based procedure, such as, for example, a luminal thickness or measurement of the presence of calcifications. In some embodiments, the treatment plan generation algorithm is configured to compare at least a subset of data regarding the catheter-based procedure against data from a previous catheter-based procedure. That is, the catheter-based information module may receive information regarding the catheter-based procedure, such as, for example, characteristics of diseased tissue (e.g., tissue compliance) or a measurement of acute valve opening or an age or a blood pressure measurement of a subject receiving catheter-based treatment, and the treatment plan generation algorithm may compare at least a subset of such received information against data from previously performed catheter-based procedures. For example, the treatment plan generation algorithm may compare at least a subset of such received information against analogous data from previously performed catheter-based procedures in order to look up previously applied configurations or settings. In some embodiments, the treatment plan generation algorithm is configured to access a collection of data regarding a plurality of previous catheter-based procedures. Such collection of data may comprise data received by the catheter-based information module regarding the catheter-based procedure as well as configuration settings applied in previously performed catheter-based procedures. Such collection of data may be organized so that data received by the catheter-based information module regarding previously performed catheter-based procedures (data comprising, e.g., measurements of tissue compliance or imaging data or assessments of diseased tissue or the like) can function as a key or an index for accessing corresponding configuration data for the corresponding catheter-based procedures. In such embodiments, the treatment plan generation algorithm may be configured to look up previously used configuration data using at least a subset of data regarding the catheter-based procedure. Embodiments that utilize such a configuration may re-apply configurations for a catheter-based procedure that were successful in previously performed catheter-based procedures.

In some cases, embodiments may comprise a treatment plan algorithm configured to extrapolate relevant configuration data, i.e., when data received by the catheter-based information module is not identical to data available in a collection of data regarding a plurality of previous catheter-based procedures. Any convenient extrapolation-based method may be applied, such as linear extrapolation. For example, in a case where a data measurement (e.g., a tissue compliance measurement) collected in connection with a prospective or on-going catheter-based procedure is at, for example, a midpoint between tissue compliance measurements of two previously performed catheter-based procedures, the treatment plan algorithm may generate a configuration setting for the prospective or on-going catheter-based procedure that reflects, for example, a midpoint between the corresponding settings of the two previously performed catheter-based procedures.

In embodiments, the treatment plan generation algorithm may be configured to utilize non-linear methods of generating treatment plans for catheter-based procedures. In some cases, the treatment plan generation algorithm comprises a machine learning algorithm. That is, in embodiments, the treatment plan algorithm may receive information regarding a catheter-based procedure, at least a subset of which information may comprise an input to a machine learning algorithm configured to generate a treatment plan. By “machine learning” algorithm, it is meant any convenient computer algorithm designed to automatically learn through experience. In embodiments, relevant machine learning algorithms may employ supervised learning, unsupervised learning or reinforcement learning approaches to generating a treatment plan. Relevant machine learning algorithms may employ regression and classification techniques to arrive at treatment plans. In embodiments, relevant experience used to train such a learning algorithm may comprise, for example, specifically constructed training data or previously collected configurations of (i.e., settings of) and/or data regarding (i.e., tissue compliance measurements or outcome information); i.e., training data may comprise any amount of available pre-treatment data, peri-treatment data and/or post-treatment data from any catheter-based procedure such as one or more previously performed catheter-based procedures. In some cases, the machine learning algorithm comprises a statistical model, a tree-based model, a deep learning model, an artificial neural network, a convolutional neural network, a deep learning model or the like, as such models and algorithms are known in the art. In other cases, a Gaussian algorithm is applied, such as, for example, a Gaussian Derivative Filtering algorithm, for detecting changes, e.g., changes in measurement values transmitted to the catheter-based procedure information module, and setting thresholding limits for occurrences, e.g., occurrences of measurement values exceeding threshold limits. Such algorithms are described in detail in, for example, U.S. Pat. No. 11,464,949 B2, the disclosure of which is incorporated herein by reference.

Embodiments of methods, in which the treatment plan algorithm comprises a machine learning algorithm, may further comprise training the machine learning algorithm with data regarding the catheter-based procedure. In some cases, data regarding one or more previously performed catheter-based procedures may be used to train a machine learning algorithm. For example, tissue characteristics (e.g., initial or intermediate valvular compliance) and/or outcome information (e.g., resulting valvular compliance) as well as the corresponding treatment plans, i.e., configuration settings (e.g., a device type or pulsatile energy pressure, frequency, duty cycle or duration of treatment (i.e., period of time or total number of balloon pulses applied to tissue)) may be provided to a machine learning algorithm for training the machine learning algorithm to make predictions regarding optimal or effective treatment plans. In embodiments, data used to train the machine learning algorithm may comprise data from a plurality of previous catheter-based procedures. In some embodiments, such training data, i.e., data regarding previous catheter-based procedures, comprises data from catheter-based procedures performed at a plurality of locations, including two, three, four, five, six, seven, eight, nine, ten, 100, 1,000, 10,000 or more different locations. That is, the catheter-based information module may be configured, e.g., interconnected, in order to receive data regarding catheter-based procedures from a plurality of different locations. For example, the catheter-based information module may be configured, e.g., interconnected, to a plurality of different treatment locations, such as different treatment locations within a treatment center or different treatment centers, such as a plurality of hospitals. In embodiments, such training data, i.e., data regarding previous catheter-based procedures, comprises data from catheter-based procedures performed on a plurality of different subjects, such as one or more subjects, including two, three, four, five, six, seven, eight, nine, ten, 100, 1,000, 10,000 or more subjects. In other cases, synthetic data, i.e., data that is generated other than by previously performed catheter-based procedures, may be used to train a machine learning algorithm. Such synthetic data may be generated in any convenient manner, and such may vary.

### Applying Treatment Plan:

Embodiments of methods according to the present invention further comprise configuring the catheter-based procedure based at least in part on the treatment plan. That is, in embodiments, treatment plans comprise settings or configurations for performing catheter-based procedures. Configuring a catheter-based treatment plan comprises applying such settings or configurations in connection with performing the catheter-based procedure. For example, a treatment plan may comprise a frequency setting for applying pulsatile energy to tissue. In such example, applying such treatment plan comprises applying pulsatile energy to tissue at the frequency setting specified in the treatment plan. In some cases, configuring the catheter-based procedure comprises applying an initial configuration for the catheter-based procedure. That is, a treatment plan may specify one or more configurations or settings at which a catheter-based procedure may be performed initially. Such settings for the catheter-based procedure may be left in place throughout the duration of the catheter-based procedure or may be changed based on subsequently obtained information, e.g., a response caused by the catheter-based treatment, e.g., a change (or, in some cases, a lack of an expected change) in tissue compliance, upon initially applying the treatment plan when performing the catheter-based procedure.

In some cases, configuring the catheter-based procedure comprises re-configuring the catheter-based procedure while performing the catheter-based procedure. That is, a treatment plan may specify one or more configurations or settings at which a catheter-based procedure may be performed after the catheter-based procedure has been initiated. In some cases, an initial treatment plan specifies a first set of configuration settings to be applied upon commencing the catheter-based treatment and a subsequent treatment plan specifies a second set of configuration settings to be applied while performing the catheter-based procedure. For example, a configuration for the catheter-based procedure may be changed or updated based on information obtained after initiating the catheter-based procedure, e.g., a response caused by the catheter-based treatment, e.g., a change (or absence of an expected change) in tissue compliance, after initially applying an initial treatment plan when performing the catheter-based procedure.

In embodiments, configuring the catheter-based procedure comprises selecting, for example, a treatment type, a treatment duration, a treatment intensity or a treatment frequency. Such configurations may apply with respect to an initial configuration or subsequent re-configurations of the catheter-based procedure. In some embodiments, treatment types may include any desired catheter-based procedure, such as, for example, application of pulsatile energy, e.g., using a balloon catheter system or application of pulsatile energy to a heart valve using a heart-tissue conforming element or application of pulsatile energy to a chronic total occlusion using a distal crosser unit. Other examples of treatment types include the application of static energy, e.g., static pressure, to tissue or any desired combination of static and dynamic energy, e.g., a combination of static pressure and pulsatile pressure. Still other examples of treatment types include other treatments performed using catheter-based procedures as such are known in the art, including, e.g., application of energy to tissue in the form of an electrical impulse such that a cavitation bubble, ultrasound wave, plasma bubble or other high-pressure impulse, or in the form of an optical or laser pulse such that a cavitation bubble, ultrasound wave, plasma bubble or other high-pressure impulse is generated, or in the form of heat energy or energy, for example, for cryotherapy, i.e., applying heat or cold to tissue.

In embodiments, a treatment duration comprises the amount of time a treatment is applied to tissue. In instances, the treatment duration specifies total treatment time and/or amounts of time different aspects of a treatment are applied to tissue. For example, in some cases, a treatment duration specifies an amount of time that static energy is applied to tissue and/or an amount of time a tissue is allowed to rest and/or an amount of time that pulsatile energy is applied to tissue. In some cases, the treatment duration comprises one or more specific times corresponding to durations of one or more aspects of a treatment or a range of potential treatment durations, e.g., a minimum and/or maximum treatment duration. In other cases, a treatment duration comprises the number of pulses applied from, for example, a balloon to tissue.

In embodiments, a treatment intensity comprises a measure of the intensity of a treatment applied to tissue. For examples, in cases, where a treatment comprises applying pulsatile or static energy to tissue, a treatment intensity comprises a measure of such static or pulsatile energy applied to the tissue, such as an amplitude or magnitude of the treatment energy or another measure of intensity of energy applied to tissue as such are known in the art, such as, for example, a root-mean-square measure of intensity of pulsatile energy applied to tissue. In other cases, treatment intensity comprises minimum and/or maximum amounts of energy applied to tissue. For example, in an embodiment in which pulsatile pressure is applied to tissue, a treatment intensity may comprise a minimum and a maximum amount of pressure applied to tissue. In instances, the treatment intensity comprises an overall treatment intensity and/or treatment intensities corresponding to aspects of a treatment. In some cases, the treatment intensity comprises one or more specific intensity values corresponding to intensities of one or more aspects of a treatment or a range of potential treatment intensities, e.g., a minimum and/or maximum treatment intensity.

In embodiments, treatment frequency comprises a frequency and/or a duty cycle at which treatment is applied. In some embodiments in which the catheter-based procedure comprises the application of pulsatile energy, e.g., applying pulsatile energy to tissue, such as cardiovascular tissue, such as heart valves, or stenotic arteries or chronic total occlusions, a treatment frequency comprises the frequency at which pulsatile energy is applied to tissue and/or a duty cycle for applying pulsatile energy to tissue. In some cases, the treatment frequency comprises one or more specific frequency and/or duty cycle values corresponding to frequency and/or duty cycle of one or more aspects of a treatment or a range of potential treatment frequencies and/or duty cycles, e.g., a minimum and/or maximum treatment frequency and/or duty cycle. In some cases, a treatment frequency may be related to a physiological aspect of a subject undergoing treatment, such as a frequency that is synchronized with a cardiac cycle of a subject.

In embodiments, configuring the catheter-based procedure comprises automatically adjusting a configuration of a catheter-based system. In some cases, upon communicating a configuration from a catheter-based information module for a catheter-based procedure, e.g., a treatment plan comprising a configuration setting for a catheter-based procedure, the catheter-based procedure may automatically reflect such configuration setting. For example, in the event a catheter-based information module communicates a treatment plan comprising a configuration setting (an initial configuration or a re-configuration) for a catheter-based procedure, the catheter-based procedure may automatically be configured to apply such configuration setting (in the case of an initial configuration, upon initiating treatment, or in the case of re-configuring the catheter-based procedure during treatment, upon continuing treatment). In some embodiments, the catheter-based procedure comprises presenting a change to a configuration of a catheter-based system for operator approval. That is, an operator of a catheter-based procedure may be required to approve a changed configuration or an initial configuration before the catheter-based procedure is configured accordingly. For example, the catheter-based information module may generate a treatment plan comprising proposed treatment settings; however, prior to applying such configuration settings to the catheter-based procedure, an operator is required to approve such changes to the configuration settings. In some cases, a change to a configuration comprises a range of potential configuration settings, and operator approval comprises selecting a specific configuration setting, such as a setting included within the range of potential configuration settings. Typically, an operator comprises a person controlling, monitoring, evaluating or otherwise present during a catheter-based procedure, such as a treatment provider present in a treatment room or a treatment provider present in a control room, e.g., remotely monitoring one or more treatments.

In embodiments, an operator of a catheter-based system, e.g., a physician, sets a configuration, such as the number of pulses to be applied by a balloon catheter. In such embodiments, the operator may configure such setting either by directly entering this setting directly into the catheter-based system or may do so based on a recommendation or suggestion received from a catheter-based procedure information module. Such recommendation or suggestion may be generated by a catheter-based procedure information module based on imaging results or other input information, i.e., information received by it based on, for example, imaging results of the relevant tissue or aspects of the catheter-based system.

In some embodiments, after access is gained (i.e., a procedure is initiated such that, for example, aspects of a catheter-based system, e.g., a distal region of a catheter, has access to a relevant tissue of a subject, i.e., is introduced into a subject), catheter location is set (i.e., a catheter or hearth-tissue-conforming element or distal crosser unit or other relevant aspect of the catheter-based system is located at a relevant location of a subject, such as proximal to a heart valve, for example), primed (i.e., fluid present in the catheter-based system is primed), and otherwise prepared to apply treatment to a relevant tissue of a subject, an operator, e.g., a physician, controls the catheter-based system (e.g., taps a treatment button once) in order to initiate treatment. Once initiated, the catheter-based system, in conjunction with the catheter-based procedure information module (i.e., based at least in part on treatment plan information provided by the catheter-based procedure information module), controls treatment administration including, for example, delivering an appropriate number of pulses of energy to relevant tissue of a subject at an appropriate (potentially variable) energy. As treatment continues, the catheter-based system, in conjunction with the catheter-based procedure information module (i.e., based at least in part on information transmitted to the catheter-based procedure information module), checks compliance change for any calcium ruptures, checks burst conditions or other failure modes, and continues administration of treatment until the pre-described pulse cycles are delivered. In some cases, treatment plan information or one or more configuration settings are periodically or continuously updated to the catheter-based system from the catheter-based procedure information module based on information transmitted to and received from the catheter-based procedure information module.

Some embodiments are configured to support continuity of treatment. For example, for continuity of treatment, an operator or the catheter-based system in connection with the catheter-based procedure information module, in either case, utilizing, for example, imaging results, can be relied up to confirm whether a previously treated lesion is being treated again or, instead, a new lesion is being treated with the same balloon. In such embodiments, if the same lesion is being treated, the catheter-based system in connection with the catheter-based procedure information module may be configured to continue treatment where treatment was previously left off during a previous treatment (i.e., a treatment plan can be generated by the catheter-based procedure information module that reflects configuration settings corresponding to where treatment was previously left off during a previous treatment). In such embodiments, if instead a different lesion is being treated, the treatment plan (e.g., generated by the catheter-based procedure information module) can start from the beginning or can be reset to another configuration.

### Catheter-Based Procedures:

Embodiments of the present invention may comprise any desired catheter-based procedure, as such are known in the art. In some cases, the catheter-based procedure comprises a method of imparting pulsatile energy to an internal luminal tissue location, including, for example, applying a pulsatile balloon catheter system. Further details regarding such catheter-based procedures which may be employed in connection with the methods and systems described herein are provided in U.S. Application No. 63/274,832, the disclosure of which is incorporated herein by reference. In other cases, the catheter-based procedure comprises a method of crossing a total occlusion, including, for example, applying a microcatheter system for crossing total occlusions. Further details regarding such catheter-based procedures which may be employed in connection with the methods and systems described herein are provided in U.S. Application No. 63/238,381, the disclosure of which is incorporated herein by reference. In still other cases, the catheter-based procedure comprises a method of imparting pulsatile energy to cardiovascular tissue, including, for example, applying a system for imparting pulsatile energy to heart valve tissue. Further details regarding such catheter-based procedures which may be employed in connection with the methods and systems described herein are described in U.S. Application Ser. No. 63/346,703 titled “Systems and Methods for Treating Cardiovascular Tissue” and filed on event date herewith (Attorney Docket No. AVSI-004PRV); the disclosure of which is incorporated herein by reference. Embodiments of the present invention may comprise catheter-based procedures for implanting devices. For example, embodiments may comprise catheter-based procedures for implanting devices such as a stent, such as a self-expanding stent, or a replacement heart valve, such as an artificial heart valve or a donor heart valve. Such embodiments may be applied such that a treatment plan relates to guidance intended to influence and/or predict the desired behavior of the device, such as an implanted device. For example, in the case of a catheter-based procedure for deploying a stent, treatment plan information may relate to predictions regarding how such stent will respond when deployed, e.g., will it jump forward when using a self-expanding stent or how will the stent and vessel tortuosity respond when a stent is deployed or will the stent cause an edge dissection or will the stent fully expand or does the vessel need more preparation. In the case of a catheter-based procedure for deploying a replacement heart valve, treatment plan information may relate to predictions regarding how the replacement heart valve will seat itself and function.

### Sensors:

In certain embodiments, a catheter-based system configured for performing aspects of catheter-based procedures according to embodiments of the invention (including, for example, heart-tissue-conforming elements or angioplasty balloons or catheters, for example), may comprise one or more sensors (e.g., pressure, temperature, volume sensor or the like) configured to acquire data from one or more locations throughout the catheter-based system prior to performing, while performing, and/or subsequent to performing, the catheter-based procedure. Such data may comprise information communicated to and/or from the catheter-based procedure information module, in embodiments of the present invention. In such embodiments, the sensors are configured to transmit to and/or receive information from a controller or another aspect of a catheter-based system, in each case, so that such information can be communicated to and/or from a catheter-based information module, or, in other cases, the sensors may be configured to communicate information directly to and/or from a catheter-based procedure information module. In such instances, data collected from sensors may be fused in order to evaluate behavior of the catheter-based system or tissue, e.g., cardiovascular tissue, on which the catheter-based procedure is conducted, that occurs at, for example, different frequencies or amplitudes of pressure applied to aspects of the catheter-based system, such as, for example, an angioplasty balloon or a heart-tissue-conforming element or angioplasty balloon or catheter, for example. A controller or other aspect of the catheter-based system or a catheter-based procedure information module may be configured to read data from sensors and adjust the treatment applied via the catheter-based procedure, e.g., the treatment provided to cardiovascular tissue, in the form of a feedback and/or a feedforward loop. The controller or other aspect of the catheter-based system or the catheter-based procedure information module may also be configured to determine a more optimal treatment profile or treatment plan (i.e., system configurations, such as, pressure, frequency and/or duty cycle configurations, for implementation by the catheter-based system to address disease conditions, e.g., calcifications present on cardiovascular tissue or other disease conditions in relevant tissue). That is, the catheter-based procedure information module may generate a revised treatment plan based on information, i.e., sensor data, collected while performing the catheter-based procedure. The controller or other aspect of the catheter-based system or the catheter-based procedure information module may also be configured to adjust the treatment profile such that the treatment is performed according to formerly determined, optimal treatment profile. Any convenient commercially available sensors (e.g., pressure, temperature, volume sensor or the like) may be utilized when performing the catheter-based procedure, i.e., utilized in the catheter-based system and integrated into aspects of the system as needed based on the sensor. Any convenient commercially available means of communicating data collected by the sensors to a controller or directly to a catheter-based procedure information module may be utilized in connection with practicing aspects of the present invention, e.g., wired or wireless data connections, e.g., network connections, dedicated communication wires or busses or the like.

### Robotic Aspects:

Another aspect of embodiments of the present invention relates to robotic treatment of diseased tissue, e.g., cardiovascular tissue. In one configuration, a catheter-based system for performing a catheter-based procedure in connection with practicing an embodiment of the invention is located at a treatment location, i.e., a location where such system is used to treat a subject, such as a procedure room or treatment room or operating room. Connected to the treatment location is a control room comprising system controls and/or system results, e.g., data collected by sensors of the catheter-based system as well as an embodiment of a catheter-based procedure information module or access to, e.g., a connection, such as a network connection, to, a catheter-based procedure information module of the present invention. The control room may be operably connected to the components of the catheter-based system present at the treatment location via any convenient connection, such as a wired or wireless data connection. The control room may include various displays for displaying information related to a catheter-based procedure using a catheter-based system such as for imaging or device sensors. Additionally, the control room may include a treatment controller such that an operator may be able to control various aspects of the treatment such as treatment initiation and device positioning, i.e., position of a catheter or angioplasty balloon or a heart-tissue-conforming element or the like with respect to tissue, e.g., cardiovascular tissue of a subject present in the treatment room. Such a treatment controller may be operably connected to a catheter-based procedure information module, such that the catheter-based information module may interface with the controller to configure, e.g., directly or via operator approval, aspects of the catheter-based system for performing the catheter-based procedure, i.e., configured to generate, store and/or transmit treatment plan information related to controlling aspects of the catheter-based system when performing a catheter-based procedure related to, for example, cardiovascular tissue of a subject. As described above, a treatment profile or treatment plan may comprise system configurations, such as, pressure, frequency and/or duty cycle configurations, for implementation by a catheter-based system to address, e.g., calcifications present on cardiovascular tissue. Other aspects of a control room may include information about a subject, e.g., a display of patient vitals or other pre- and/or peri- and/or post-treatment information.

In some cases, the control room may be a few feet from the operating table such as behind a lead curtain or other form of shielding or may be located at a distinct geographic location, such as in another building or another city or country. One or more operators may be able to interact in the control room and may be able to communicate with the subject or other operators at the treatment location.

### Visualization:

Performing catheter-based procedures of the present invention may further comprise using a system comprising marker bands present on aspects of the system, such as, for example, a catheter or an angioplasty balloon or heart-tissue-conforming element or the like. Marker bands may be affixed to different components of the system, such as, for example, to various locations on a catheter, heart-tissue-conforming element or a guidewire threaded through the catheter. Marker bands may be used to visualize the position of the system, or components thereof, such as the heart-tissue-conforming element, when applied to a subject via, for example, any convenient fluoroscopy technique. Results of visualization techniques, such as a location or configuration or orientation of a catheter-based system for performing catheter-based procedures of the present invention may comprise data communicated to and/or from a catheter-based information module. In instances, the catheter-based information module may be configured to evaluate visualization information, e.g., may be configured to perform image processing to assess any relevant aspect of the catheter-based procedure, such as, for example, changes in tissue characteristics resulting from performing the catheter-based procedure or changes in location of a catheter-based system with respect to a subject while performing the catheter-based procedure.

Marker bands used in connection with performing catheter-based procedures may be any convenient, readily available, off the shelf marker band capable of being affixed, for example, swaged, crimped or heat bonded or welded or bonded, to components of the catheter-based system. Marker bands of interest may be polymer bands laden with gold or platinum or tungsten or another material that facilitates visualization, such as visualization via fluoroscopy. Marker bands may be visualized via fluoroscopic or radioscopic visualization techniques.

Embodiments of the present invention may be configured to take into account the anatomical location of a catheter-based procedure in order to fuse imaging data with treatment data (e.g., fusing imaging data with other data obtained before, during or after treatment). Such embodiments may be used for overlaying pre- and post-treatment results and determining outcomes. Such embodiments may comprise registration of pre- and post-treatment imaging data.

Certain embodiments of the present invention may apply imaging techniques, such as, for example, optical coherence tomography (OCT) or intravascular ultrasound (IVUS) imaging or fluoroscopy or other imaging modalities. In some instances, such embodiments may further employ techniques based at least in part on such imaging to automatically measure calcium structure (i.e., calcium eccentricity, calcium thickness and/or calcium length) present in relevant tissue, assign a score representing aspects of such calcium present in relevant tissue and generate a treatment plan, where the treatment plan includes, for example, a peak pressure, frequency and/or intravascular lithotripsy energy, based at least in part on such score. Any available technique for scoring based on imaging results may be employed, including, for example, OCT/IVUS scoring system based on Zhang, et al., Intravascular Ultrasound Versus Angiography-Guided Drug-Eluting Stent Implantation: The ULTIMATE Trial, J Am Coll Cardiol. 2018 Dec. 18; 72(24):3126-3137, doi: 10.1016/j.jacc.2018.09.013, Epub 2018 Sep. 24, PMID: 30261237, or an angiographic scoring system based on Rocha-Singh, et al., Peripheral arterial calcification: prevalence, mechanism, detection, and clinical implications, Catheter Cardiovasc Interv. 2014 May 1; 83(6):E212-20, doi: 10.1002/ccd.25387, Epub 2014 Feb. 10, PMID: 24402839; PMCID: PMC4262070; each reference is hereby incorporated herein in its entirety. In embodiments, such a scoring system may be applied for pre-treatment planning, and additional measurements can be taken during treatment or post-treatment to measure the amount of cracking of the calcium structure. To ensure an optimal treatment energy is delivered, embodiments may comprise counting cracks generated in calcium structures and further, in some cases, may comprise informing a clinician if sufficient energy has been applied to treat the lesion. Such processes may occur on a patient-by-patient basis or may be dependent on a learning algorithm, such as a cloud-based learning algorithm (i.e., pre-, peri- and/or post-treatment OCT or IVUS images are fused with treatment data to estimate the number of calcium cracks and luminal expansion that occurs). Based on such results, future treatments, i.e., treatment plans generated subsequently, with the benefit of such data, output the appropriate amount of energy to ensure that sufficient cracking occurs to achieve the most optimal luminal gain in the safest manner.

Various aspects of methods of embodiments of the invention being generally described above, aspects of methods of the invention are now further reviewed in the context of specific embodiments.

### SPECIFIC EMBODIMENTS

Aspects of the claimed invention are described in connection with embodiments of the method set forth in the figures described below for ease of illustration only and without limiting the present invention to such embodiments.

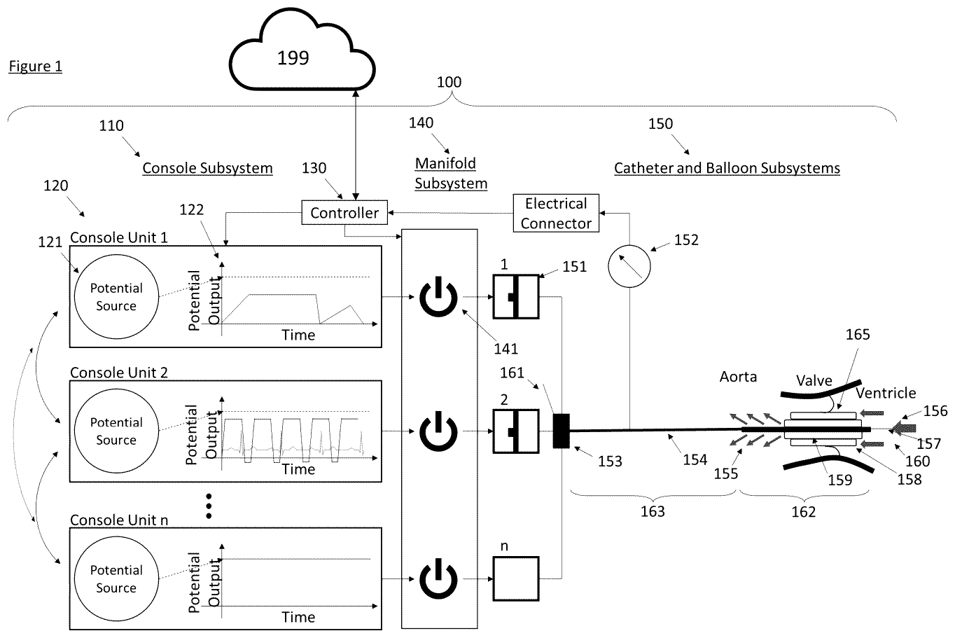

An exemplary catheter-based system for use in performing a catheter-based procedure in connection with an embodiment of the invention is schematically illustrated in FIG. 1. In the embodiment set forth in FIG. 1, the system is configured for imparting pulsatile energy to cardiovascular tissue. In some instances, the system can include a console assembly which has a potential energy source, such as a high voltage or pressure source, a regulator, e.g., for regulating the output of a pressure source, etc., and a controller, e.g., for controlling the aspects of the system. The system can also include a manifold assembly operably connected to an output of the console assembly comprising an oscillator for converting the output of the potential source into pulse energy. The system can also include a catheter assembly operably connected to an output of the manifold assembly for converting the output of the manifold assembly (i.e., first pulsatile energy), into, e.g., hydraulic oscillations or other forms of oscillatory potential energy (i.e., second pulsatile energy) and transmitting the second pulse energy via a catheter (i.e., a fluidic passage of a catheter) to a heart-tissue-conforming element. The heart-tissue-conforming element can be configured to receive oscillatory potential energy and apply pulsatile energy to cardiovascular tissue. In embodiments, the oscillatory potential energy acts to drive aspects of the heart-tissue-conforming element, such as, for example, one or more balloon angioplasty oscillations or movement of radial members to bend or flex cardiovascular tissue. A controller can control the frequency, duty cycle and/or amplitude of the outputted energy from the potential source of the console assembly as well as the oscillator of the manifold assembly, e.g., based on configuration settings included in a treatment plan. A connector of the catheter assembly can convert energy outputted by the manifold assembly into, e.g., hydraulic oscillations, that thereby generate oscillations in aspects of a heart-tissue-conforming element.

FIG. 1 depicts a schematic view of an exemplary embodiment of a system 100 for imparting pulsatile energy to cardiovascular tissue according to the present invention. The exemplary system 100 for performing a catheter-based procedure depicted in FIG. 1 includes three assemblies: a console assembly (i.e., console subsystem) 110, which may comprise one or more console units 120; a manifold assembly (i.e., manifold subsystem) 140, which may comprise one or more subunits including an oscillator 141; and catheter assembly (i.e., catheter and balloon subsystems) 150, which may comprise one or more connectors 151, as well as catheter 154 with a fluidic passage (not shown) and heart-tissue-conforming element 165. Each connector 151 of catheter assembly 150 is connected to an oscillator 141 of manifold assembly 140 and one or more connector-to-catheter transition hubs 153 for transmitting pulsatile energy (i.e., second pulse energy) to heart-tissue-conforming element 165 via catheter 154.

Console assembly 110 may comprise one or more console units 120. When the console assembly comprises a plurality of console units, such console units may be combined into a single physical component (i.e., housing) or separated into a plurality of housings, e.g., one housing per each console unit. In each case, console units are configured to be operated independently of each other, i.e., are capable of being controlled independently, whether the console units are present in a single housing or multiple housings. Console unit 120 includes a potential source 121 for generating energy for transmission to manifold assembly 140, a potential regulator (not shown) and controller 130. The output from potential source 121 may include regulated 122 or unregulated potential energy, such as that from high-pressure fluid or voltage. A potential regulator may be used to modify potential energy output from potential source 121 into a form that can be transmitted and further manipulated by manifold assembly 140, i.e., such that oscillator 141 can generate pulse energy from energy transmitted from potential source 121. Multiple console units 120 (i.e., console units numbered 1 through n) can be included in console assembly 110 and can operate substantially in parallel (i.e., independently) to generate multiple potential outputs 122 for transmission to multiple oscillators 141. In some instances, multiple console units 120 can be configured to generate multiple potential outputs 122 in cases where treatment of cardiovascular tissue comprises applying different configurations of pulsatile energy to cardiovascular tissue, for example, where the heart-tissue-conforming element comprises a plurality of balloons configured to be inflated independently potentially at different frequencies, duty cycles and/or amplitudes. In such instances, different potential outputs 122 may separately be applied to cardiovascular tissue, e.g., via different balloons or other aspects of the heart-tissue-conforming element or at different times (i.e., where one potential output is operably connected to a balloon at a first time and subsequently another potential output is operably connected to the balloon at a second time). In other instances, potential output 122 from multiple console units 120 may be combined. In still other instances, multiple console units 120 can be configured to generate multiple potential outputs 122 including different forms of potential energy (e.g., high pressure or voltage) in cases where treatment of cardiovascular tissue requires application of different forms of energy.

Console assembly 110 further comprises controller 130 configured to receive input from at least one of console assembly 110, manifold assembly 140 and catheter assembly 150. Controller 130 is further configured to be operably connected to catheter-based procedure information module 199 such that data regarding the catheter-based procedure performed by system 100 can be communicated to and/or from catheter-based procedure information module 199. In FIG. 1, controller 130 is shown receiving input from catheter assembly 150, i.e., sensor 152 of catheter assembly 150. Sensor 152 can comprise any sensor configured to sense any relevant characteristic of catheter assembly 150 capable of detection. For example, sensor 152 may comprise a pressure transducer configured to measure a pressure within catheter assembly 150, such as, for example, a pressure of a fluid channel of catheter 154 or a pressure of an aspect of heart-tissue-conforming element 165 such as an angioplasty balloon; alternatively, sensor 152 may comprise a volume sensor, configured to measure a volume of fluid present in, for example, a balloon. In instances, controller 130 can be configured to receive input from a plurality of sensors, including sensors configured to measure any relevant aspect of system 100 or the environment in which system 100 is applied (e.g., pressure sensor, temperature sensor, volume sensor or the like) and may be configured to capture data from any location throughout system 100, including, for example, one or more locations of system 100, e.g., locations on heart-tissue-conforming element 165, catheter 154, connector-to-catheter transition hubs 153, connector 151, oscillator 141 or console unit 120. In general, in embodiments, sensors can be configured at any desired location of the system 100 to gather any desired information regarding use of the system, e.g., in connection with a treatment procedure. In other instances, controller 130 can be configured to receive input from user inputs such as buttons or switches for specifying treatment options (e.g., system pressures, frequencies, duty cycles, etc.) or amending or updating treatment options or configurations, e.g., confirming or revising aspects of a treatment plan generated by, and communicated from, catheter-based procedure information module 199. In FIG. 1, controller 130 receives input from pressure transducer 152 of catheter assembly 150 and, based at least in part on such input, generates a control signal for controlling aspects of console assembly 110, such as, for example, a magnitude of potential output 122, i.e., via an active regulator (not shown) used to adjust a magnitude of potential output 122 (e.g., an output pressure).

In embodiments as illustrated schematically in FIG. 1, an output of console assembly 110 is operably connected to manifold assembly 140, such that energy transmitted (i.e., regulated potential output 122) from potential source 121 of console unit 120 is transmitted to oscillator 141 of manifold assembly 140. Oscillator 141 is configured to generate pulsatile or static energy from energy transmitted from potential source 121 (i.e., regulated potential output 122). In instances, oscillator 141 may include a solenoid valve (not shown) configured to either allow or interrupt transmission of energy to catheter assembly 150. In other instances, oscillator 141 may include any applicable electrical, e.g., electrical solenoid, optical or mechanical switch, as such are known in the art.

In FIG. 1, controller 130 is shown connected to manifold assembly 140. In instances, oscillator 141 may be configured so that an oscillation frequency and/or duty cycle may be controlled by controller 130, e.g., such that controller 130 controls a position or other aspect of the behavior of a solenoid of oscillator 141.

In embodiments as illustrated schematically in FIG. 1, an output of oscillator 141 of manifold assembly 140 is operably connected to catheter assembly 150. In particular, an output of oscillator 141 is connected to an input of connector 151. Connector 151 is configured to transduce potential energy such as, for example, pneumatic pressure (i.e., a first pulse energy), generated by oscillator 141, to a second potential energy such as, for example, hydraulic pressure (i.e., a second pulse energy). System 100 shown in FIG. 1 includes multiple connectors 151 (connectors 1 through n), one connector 151 corresponding to each oscillator 141. Outputs of connectors 151 are operably connected to connector-to-catheter transition hub 153 configured to enable potential energy output of connectors 151 (i.e., second pulse energy) to be input into catheter 154, e.g., one or more fluidic channels (not shown) of catheter 154. In some cases, catheter assembly 150 comprises more than one catheter 154, or catheter 154 comprises more than one fluidic channel internal or external to catheter 154, i.e., such that different aspects of heart-tissue-conforming element 159 may be independently operated (e.g., such that different angioplasty balloons or radial members comprising heart-tissue-conforming element 159 may be independently pressurized or inflated and deflated).

In catheter assembly 150 of system 100, catheter 154 includes a channel dedicated for a guidewire so that catheter 154 and heart-tissue-conforming element 159 can be navigated to a cardiovascular tissue treatment site via a standard over-the-wire guidewire approach. Connector-to-catheter transition hub 153 is configured to include guidewire exit port 161 for a proximal region of a guidewire threaded through a guidewire channel of catheter 154. Guidewire exit port 161 is opposite guidewire entrance port 157 present at a relatively distal region of heart-tissue-conforming element 165. For illustrative purposes only, in order to highlight the location of guidewire exit port 161 relative to other components of system 100, guidewire exit port 161 is depicted as oriented away from the longitudinal axis of catheter 154. In embodiments, guidewire exit port 161 is oriented in a manner that is parallel to the longitudinal axis of catheter 154 (and therefore parallel to the longitudinal axis of the guidewire channel within catheter 154), in order to avoid any unnecessary bends in a guidewire present in system 100. As described above system 100 may be configured with respect to the guidewire so that system 100 is an over-the-wire, rapid exchange, monorail or the like system.

In the system illustrated schematically in FIG. 1, heart-tissue-conforming element 165 is present at a distal region of catheter 154. In system 100, heart-tissue-conforming element 165 is configured to conform to cardiovascular tissue comprising an aortic heart valve, such that heart-tissue-conforming element 165 spans the leaflets of the aortic valve with a distal region of heart-tissue-conforming element 165 present on the ventricle side of the aortic valve and a proximal region present on the aortic side of the aortic valve. Heart-tissue-conforming element 165 includes a plurality of angioplasty balloons and other features (e.g., locating elements or nubs or the like) configured to engage aspects of the aortic heart valve. The angioplasty balloons of heart-tissue-conforming element 165 include one or more outer commissure balloons (e.g., winged balloons) 158 and one or more inner valvuloplasty balloons (e.g., mid-radius balloons) 159. Angioplasty balloons 158,159 of heart-tissue-conforming element 165 may be independently operably (i.e., capable of pressurization and depressurization independent of whether other balloons are pressurized or depressurized).

Outer commissure balloon 158 is present at a relatively outer radial distance from the longitudinal axis of heart-tissue-conforming element 165 sufficient so that outer commissure balloon 158 can engage with (i.e., apply pulsatile energy to) an aortic valve commissure, e.g., to disrupt calcium formations in an aortic valve commissure that inhibits proper valve function. Outer commissure balloon 158 may comprise non-compliant/compliant material. Heart-tissue-conforming element 165 may comprise multiple outer commissure balloons 158 configured so that outer commissure balloons 158 engage each valve commissure of the aortic valve, for example, comprising three outer commissure balloons 158 to engage each aortic valve commissure.

Outer commissure balloon (e.g., winged balloon) 158 may be configured to be independently operable, i.e., independently pressurized and depressurized, from a second outer commissure balloon and from inner valvuloplasty balloon 159, by, for example, being pressurized by hydraulic pressure transmitted via separate fluidic chambers of catheter 134. Alternatively, outer commissure balloons 158 may be configured so that they are pressurized and depressurized simultaneously, by, for example, being pressurized by hydraulic pressure transmitted via a single fluidic channel of catheter 134 that feeds multiple outer commissure balloons 158.

Inner valvuloplasty balloon (e.g., a mid-radius balloon) 159 is present at a relatively inner radial distance from the longitudinal axis of heart-tissue-conforming element 165 sufficient so that inner valvuloplasty balloon 159 can engage with (i.e., apply pulsatile energy to) an aortic valve leaflet, e.g., to disrupt calcium formations in an aortic valve leaflet that inhibits proper valve function. Heart-tissue-conforming element 165 may comprise multiple inner valvuloplasty balloons 159 configured so that inner valvuloplasty balloons 159 engage each valve leaflet of the aortic valve. For example, heart-tissue-conforming element 165 may comprise three inner valvuloplasty balloons 159 to engage each aortic valve leaflet or a single inner valvuloplasty balloon 159 configured so that different surfaces of the single balloon engage each aortic valve leaflet.

Inner valvuloplasty balloon 159 may be configured to be independently operable, i.e., independently pressurized and depressurized, from a second inner valvuloplasty balloon and from outer commissure balloon 158, by, for example, being pressurized by hydraulic pressure transmitted via a separate fluidic channel of catheter 134. Alternatively, inner valvuloplasty balloons 158 may be configured so that they are pressurized and depressurized all together simultaneously, by, for example, being pressurized by hydraulic pressure transmitted via a single fluidic channel of catheter 134 configured to feed multiple balloons.

In system 100 illustrated schematically in FIG. 1, catheter 154 and heart-tissue-conforming element 165 are configured to allow fluid, e.g., blood, to perfuse past heart-tissue-conforming element 165 even while applying pulsatile energy to cardiovascular tissue at a treatment site. For example, in system 100, catheter 154 and heart-tissue-conforming element 165 are configured to enable blood to be displaced, i.e., perfuse, from perfusion entrance zone 156 to perfusion exit zone 155 via one or more passageways (not shown), e.g., fluidically connecting perfusion entrance zone 156 with perfusion exit zone 155, permitting blood received from a distal region (i.e., ventricular region) to exit at a proximal region (i.e., aortic region) of heart-tissue-conforming element 165. Perfusion entrance zone 156 and perfusion exit zone 155 comprise perforations or ports (not shown) in catheter 154 or heart-tissue-conforming element 165 through which blood can flow. Such a configuration enables blood flow past heart-tissue-conforming element 165 enabling extended treatment times. Such a perfusion mechanism may further comprise a structure (e.g., valve or oscillating balloon mechanism) in catheter 154 or heart-tissue-conforming element 165 permitting fluid to flow in only one direction. Catheter 154 comprises proximal catheter section 163 and distal catheter perfusion section 162. Distal catheter perfusion section 162 comprises perfusion entrance zone 156, perfusion exit zone 155 as well as one or more passageways (not shown), e.g., fluidically connecting perfusion entrance zone 156 with perfusion exit zone 155. Proximal catheter section 163 and distal catheter perfusion section 162 may be connected by a connector (not shown).

Any convenient perfusion mechanism may be employed in the catheter 154 and heart-tissue-conforming element 165 to induce the flow of fluid, e.g., blood, through a perfusion zone, i.e., from perfusion entrance zone 156 to perfusion exit zone 155, past heart-tissue-conforming element 165, i.e., displaced from a distal region to a proximal region of heart-tissue-conforming element 165 such that heart-tissue-conforming element 165 does not completely obstruct the flow of fluid, e.g., blood, when present in, e.g., a vessel. As described above, a passive perfusion mechanism may be employed to induce fluid to flow from perfusion entrance zone 156 to perfusion exit zone 155 based on an existing pressure gradient, i.e., an existing pressure differential caused by, e.g., ventricle contraction, or an active perfusion mechanism comprising, for example, a syringe, such as a barrel syringe, configured to displace fluid through a perfusion zone from perfusion entrance zone 156 to perfusion exit zone 155 and past heart-tissue-conforming element 165.

Aspects of system 100, such as, for example, console assembly 110 (e.g., controller 130), manifold assembly 140 (e.g., oscillator 141) as well as aspects of catheter assembly 150 (e.g., connectors 151 and connector-to-catheter transition hubs 153) may be configured to be reusable. In instances, aspects of system 100, such as, for example, catheter 154 or heart-tissue-conforming element 159 may be configured to be used a single time (e.g., such that it is disposable). The terms “reusable” and “disposable” as employed here and elsewhere throughout the description are used for convenience in describing, for example, catheter-based systems, such as exemplary system 100 illustrated in FIG. 1. However, systems for use in catheter-based procedures are not so limited. As such, any part of a system used in connection with a catheter-based procedure of the present invention may be configured for one time use or for use multiple times, as desired.

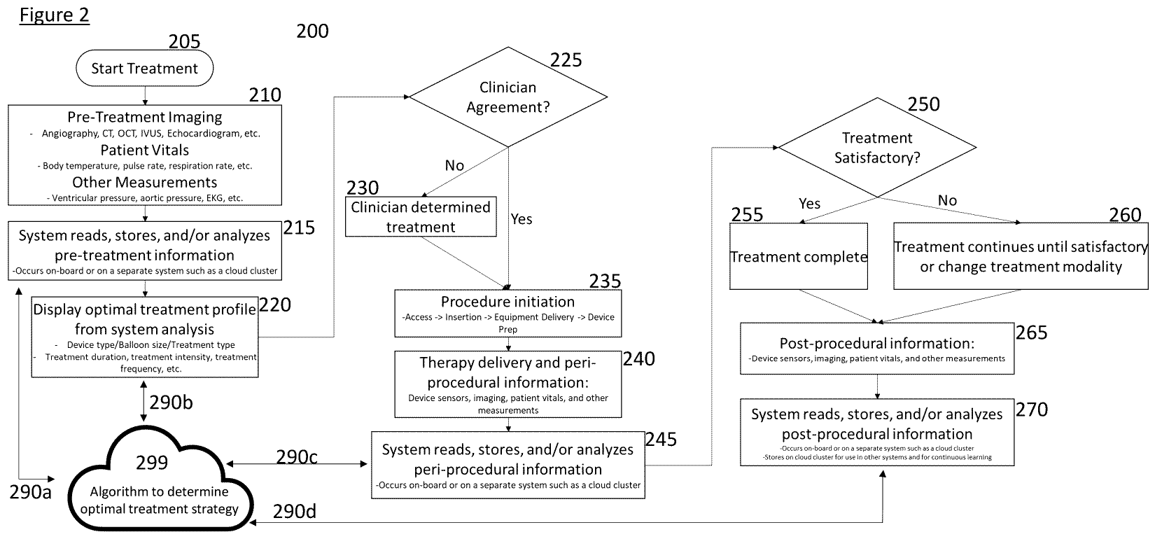

FIG. 2 illustrates a flow diagram 200 for performing a method according to some aspects of the present disclosure pertaining to performing a catheter-based procedure on a subject and communicating data regarding the catheter-based procedure to an/or from a catheter-based procedure information module. The exemplary catheter-based procedure performed in connection with flow diagram 200 may comprise using a catheter-based system such as, for example, catheter-based system 100 set forth in FIG. 1, e.g., to apply pulsatile energy to cardiovascular tissue.

The process begins at block 205 of flow diagram 200, in which treatment is started, i.e., the method according to some aspects of the present disclosure is initiated. From block 205, the process continues to block 210. At block 210, prior to performing a catheter-based procedure on a subject, pre-treatment data regarding the subject is collected. Pre-treatment data collected at block 210 may comprise, for example, imaging data, vital sign data or other measurements pertaining to any relevant aspect of the catheter-based procedure, e.g., pertaining to the subject or to the catheter-based system used to perform the procedure. Imaging data, as described above, may include, for example, one or more of angiography imaging, computed tomography imaging, optical coherence tomography, intravascular ultrasound imaging, echocardiogram imaging or any other desired imaging technique that may be used in connection with a catheter-based procedure, as such are known in the art. In some cases, imaging data is used to visualize aspects of diseased tissue, which the catheter-based procedure is intended to address. In other cases, imaging data is used to visualize aspects of the subject, e.g., the subject's anatomy, or aspects of the catheter system. Vital sign information, as described above, may include, for example, a body temperature, a pulse rate, a respiration rate or any other physiological measurement of the subject relevant to the catheter-based procedure, as such is known in the art. Other measurements, i.e., pre-treatment measurements, as described above, may include, for example, measurements of, a ventricular pressure, an aortic pressure, coronary artery pressure, results of an electrocardiogram or any other relevant measurement related to the subject or to the catheter-based procedure, e.g., a measurement of an aspect of a catheter-based system for performing the catheter-based procedure. Pre-treatment information, such as that described above, collected at block 210, may be collected by a catheter-based system used in connection with performing a catheter-based procedure, such as system 100 of FIG. 1, for example, or may be collected using other devices or systems capable of collecting relevant pre-treatment data and communicating collected pre-treatment data to either or both of such catheter-based system for performing the catheter-based procedure or directly to catheter-based information module 299. From block 210, the process continues to block 215.

At block 215, pre-treatment data collected at block 210 is communicated 290a to catheter-based procedure information module 299. Pre-treatment data is communicated 290a to catheter-based procedure information module 299 so that the pre-treatment data can be read, stored and analyzed by catheter-based procedure information module 299. Catheter-based information module 299 is depicted as being located at a remote location, e.g., a cloud-based configuration such as on a cloud cluster available over and accessed via a network connection, e.g., via any convenient internet connection, such as a wired or wireless connection; however, in other cases, catheter-based procedure information module 299 may be integrated into the catheter-based system used to perform the catheter-based procedure. In the embodiment, analyzing pre-treatment data by the catheter-based procedure information module 299 comprises applying a treatment plan algorithm configured to generate a treatment plan (i.e., a potential configuration of an aspect of performing a catheter-based procedure as described above)—in this case, an initial treatment plan—based at least in part on the pre-treatment data communicated to catheter-based procedure information module 299. As described above, a treatment plan algorithm as applied by catheter-based information module 299 may comprise a look-up algorithm, e.g., an algorithm wherein pre-treatment data is used to access a data store (e.g., a database) of previously performed catheter-based procedures, in some cases with comparable pre-treatment data such that a corresponding treatment plan may be applied. In other cases, a treatment plan algorithm as applied by catheter-based information module 299 may comprise one or more machine learning algorithms trained to generate a treatment plan based at least in part of the pre-treatment data collected in block 210. At block 215, the treatment plan algorithm is further configured to store pre-treatment data received in block 210 to a data store, such as a cloud-based data store for, e.g., subsequent access (i.e., look-up) by the treatment plan algorithm or for use in training or refining or further training a machine learning algorithm of the treatment plan algorithm. At block 215, a treatment plan (i.e., a treatment profile) is generated based at least in part of the pre-treatment data collected at block 210. The resulting treatment plan comprises one or more initial configuration settings of the catheter-based system used for performing the catheter-based procedure.

Any convenient technique may be applied in connection with the catheter-based information module 299 receiving 290a and processing pre-treatment data collected at block 210 (as well receiving 290c and processing peri-treatment data collected at block 240 and 245 and receiving 290d and processing post-treatment data collected at block 265 and 270). That is, any convenient technique may be applied such that data received by the catheter-based information module 299 is meaningful with respect to, e.g., generating a treatment plan and/or updating a database of catheter-based procedure information, such as configuration settings. In some cases, such techniques include physician tagging of relevant data, conducting database comparison and/or lookup in connection with data received by the catheter-based information module 299. In some cases, treatment data may be presented to a clinician or other trained personnel, such as medically trained personnel, for tagging or classification or identification of relevant features or aspects of collected data. In some cases, such data identification process may be gamified such that in exchange for processing data (e.g., tagging or classifying or identifying relevant features of data collected in connection with a catheter-based treatment) receive a reward, such as a form of payment or credit. Other techniques for leveraging collective expertise of trained personnel in order to build a population of well tagged/classified/identified data may be applied, such as, for example, peer review programs for group based assessment of procedural skills, such as those based on S. Spehar, et al., Blinded Cross-Institutional Peer Review Across Michigan: An Innovative Initiative for Improving Percutaneous Coronary Intervention (PCI) Appropriateness and Quality, J Am Coll Cardiol. 2022 March, 79 (9_Supplement) 615, incorporated herein by reference.

From block 215, the process continues to block 220.

At block 220, the results of analyzing the pre-treatment data, i.e., applying a treatment plan algorithm, by catheter-based information module 299, are displayed. For example, such results may be displayed on a monitor or screen configured for an operator or treatment provider to review. In particular, at block 220, information regarding the treatment plan, i.e., an optimal treatment profile, is communicated 290b from catheter-based procedure information module 299 and displayed for review by an operator. Such treatment plan communicated 290b from catheter-based procedure information module 299 is displayed for operator approval on a display operably connected to the catheter-based system, i.e., for performing the catheter-based procedure. Such operator may be a treatment provider, or a person otherwise assisting with performing the catheter-based procedure, present at a treatment location or at a control location, e.g., control room, removed from the catheter-based procedure location. Elements of the treatment plan communicated 290b from catheter-based procedure information module 299 at block 220 may include a device type, including a balloon type (i.e., size, shape, compliance characteristics, material, other features, such as locating nubs, etc.), a treatment type (i.e., pulsatile energy, static energy, application of pressure or other forms of energy), a treatment duration (i.e., an amount of time that a catheter-based procedure, or aspects thereof, is applied), a treatment intensity (i.e., a minimum and/or a maximum magnitude of energy applied via the catheter-based procedure), a treatment frequency (i.e., a frequency of pulsatile energy applied via the catheter-based procedure or a duty cycle of the catheter-catheter based procedure) or any other configuration setting relevant to a catheter-based procedure, as known to those skilled in the art. From block 220, the process continues to block 225.

At block 225, the treatment plan (i.e., the initial treatment plan), or aspects thereof, generated by the catheter-based procedure information module 299 is presented for approval to an operator or treatment provider responsible for controlling aspects of the catheter-based procedure, e.g., a clinician at or near the treatment location, e.g., control room or treatment room. The catheter-based system for performing the catheter-based procedure may comprise any convenient display or monitor for presenting the treatment plan to the operator. The catheter-based system for performing the catheter-based procedure may further comprise one or more input devices for the operator to confirm agreement with the proposed treatment plan, e.g., by clicking on a confirm button using a mouse or touchpad device such as a touchscreen.

From block 225, the process moves to block 235 in the event the operator confirms agreement with the treatment plan. Alternatively, in the event the operator indicates a desire to change an aspect of the treatment plan, the process moves to block 230 where the operator may input an alternative treatment plan, e.g., one or more alternative configurations or settings comprising the treatment plan, referred to as a clinician determined treatment (e.g., the operator may indicate that a different balloon type will be used in performing the catheter-based procedure). That is, from block 225, the process moves to block 230 in the event the operator inputs an alternative configuration. Any relevant aspect of the treatment plan may be re-configured by the operator at block 230; i.e., the catheter-based system may be configured so that the operator has the ability to override any configuration setting of the catheter-based procedure, such that the treatment plan effectively comprises an initial suggested configuration. Upon entering alternative configuration settings at block 230, the process moves to block 235.

At block 235, an initial treatment plan, i.e., an initial configuration for the catheter-based procedure, having been finalized via operator confirmation or amendment at blocks 225 and 230, i.e., the initial treatment plan is ready for application in a catheter-based procedure, and comprises either a treatment plan generated by the catheter-based procedure information module 299 or a treatment plan modified or otherwise updated by an operator, i.e., clinician, at block 230. Applying the configurations included in the treatment plan (e.g., device type, balloon type, etc.), the catheter-based procedure is initiated at block 235. By initiating the catheter-based procedure at block 235, it is meant that the catheter-based device, i.e., catheter and balloon or other treatment device affixed to a distal end of the catheter, are selected and prepared for treatment, i.e., application to a subject, as is known to those skilled in the art. Initiating the catheter-based procedure further comprises accessing a treatment site, e.g., via insertion of a catheter and catheter device in the subject. For example, in a case where the catheter device comprises a heart-tissue-conforming element, an appropriately sized and configured heart-tissue conforming element is selected, e.g., a heart-tissue conforming element with winged balloons for addressing calcifications in heart valve commissures, and the heart-tissue conforming element is positioned near a diseased heart valve of the subject. Upon initiating the procedure at block 235 the process moves to block 240.

At block 240, the catheter-based procedure proceeds such that treatment is deployed by, for example, configuring the catheter-based procedure to deliver energy, such as pulsatile energy, to tissue, i.e., cardiovascular tissue of a subject. That is, at block 240, the catheter-based procedure delivers therapy, for example, in the form of delivering energy to tissue (e.g., for disrupting calcifications within tissue of the subject). For example, at block 240, in instances where the catheter-based procedure comprises applying pulsatile energy to tissue, such pulsatile energy is applied to and delivered to tissue.

In addition, at block 240, peri-treatment data (i.e., peri-procedural data) regarding the catheter-based procedure is collected. For example, sensors, including sensors internal and external to the catheter-based system for performing the catheter-based procedure, are configured to collect data regarding the treatment and/or the subject. As described above, peri-treatment data may comprise information derived from device sensors (e.g., pressure or volume measurements), imaging data (e.g., ultrasound images indicating the location of aspects of a catheter device relative to tissue or images of tissue), subject vitals (e.g., pulse, respiration rate, blood pressure, etc.) or other measurements relevant to the catheter-based procedure. Depending on the type of treatment applied, peri-treatment data may comprise estimates of changes in tissue compliance for evaluating the effectiveness of the catheter-based procedure or other measurements that can be used for evaluating the effectiveness of the catheter-based procedure. Upon collecting peri-treatment data, the process moves to block 245.

At block 245, the peri-treatment data is communicated 290c to catheter-based procedure information module 299 so that such peri-treatment data can be read, stored and/or analyzed by catheter-based procedure information module 299, present locally at or near the treatment location, including, in some cases, integrated into the catheter-based system used to apply the catheter-based procedure, or present remotely, e.g., comprising a distributed system available over network connections, e.g., a cloud-based configuration. Analysis of peri-treatment data performed by catheter-based procedure information module 299 may comprise computations or look-ups to reference or historical data for evaluating the effectiveness of the catheter-based procedure. In some cases, analysis of peri-treatment data performed by catheter-based procedure information module 299 comprises comparing the instant peri-treatment data with historical peri-treatment data or applying a machine learning algorithm to evaluate peri-treatment data, such as to the evaluate the effectiveness of the catheter-based procedure based on aspects of the peri-treatment data. Upon analyzing peri-treatment data by catheter-based procedure information module 299, results of such analysis of peri-treatment data by catheter-based procedure information module 299 are communicated 290c to the catheter-based system and the process moves to block 250.

At block 250, at least a subset of the results of analysis of peri-treatment data by catheter-based procedure information module 299 are presented to the operator of the catheter-based procedure or treatment provider for confirmation of whether the results of applying the catheter-based procedure are satisfactory, and therefore complete, or unsatisfactory and therefore the catheter-based procedure should be continued to apply further treatment. Such results may be presented to the operator using any convenient display or monitor, for example, and the operator may indicate whether treatment is satisfactory using any convenient input device, such as, for example, a mouse or touchpad or touchscreen.

In the event the operator indicates that the results of treatment are satisfactory, the process moves to block 255, where treatment is completed, i.e., the catheter-based procedure ceases applying energy to tissue, e.g., cardiovascular tissue of a subject, and the process moves to block 265. In the event the operator indicates that treatment is not satisfactory (i.e., the catheter-based procedure should continue to apply treatment to diseased tissue), the process moves to block 260.

At block 260, treatment, i.e., application of the catheter-based procedure, is continued, i.e., the catheter-based procedure continues to deliver energy to tissue, under the same or a revised treatment plan, as generated by catheter-based procedure information module 299 with or without operator amendment to the configuration settings relevant to the catheter-based procedure, i.e., a treatment plan that is a revised treatment plan by the catheter-based procedure information module 299 and/or by the operator of the catheter-based procedure.

In some cases, at block 260, additional peri-treatment data is collected and communicated to catheter-based procedure information module, analogous to the steps shown in blocks 240 and 245. Further, at block 260, in some cases, a revised treatment plan is communicated from catheter-based procedure information module 299 in order to revise the configuration of the catheter-based procedure; i.e., a treatment modality (e.g., treatment type, device type, balloon type, treatment duration, treatment intensity, treatment frequency, etc.) may be modified in connection with continuing the catheter-based procedure. In addition, at block 260, subsequent peri-treatment data may be collected and analyzed such that an operator may indicate, based on such results, whether treatment is satisfactory. Upon an indication that treatment is satisfactory, or upon the occurrence of another treatment-end signal, such as completion of a treatment duration, the process moves to block 265.

At block 265, post-treatment data (i.e., post-procedural data) regarding the catheter-based procedure is collected. For example, sensors, including sensors internal and/or external, to the catheter-based device are configured to collect data regarding the catheter-based treatment. As described above, post-treatment data may comprise information from device sensors (e.g., pressure or volume measurements), imaging data (e.g., ultrasound images indicating the location of aspects of a catheter device relative to tissue or images of tissue), subject vitals (e.g., pulse, respiration rate, blood pressure, etc.) or other measurements relevant to the catheter-based treatment. Post-treatment data also includes outcome information regarding the catheter-based procedure. That is, post-treatment data may include outcome information comprising, for example, data regarding acute valve opening, tissue compliance, such as valvular compliance, intravascular pressure or external pressure, in each case, upon completion of the catheter-based procedure. Depending on the type of treatment applied, post-treatment data may comprise estimates of changes in tissue compliance for evaluating the effectiveness of the catheter-based procedure or any other measurement relevant to the catheter-based procedure for use evaluating the effectiveness of the catheter-based procedure. Upon collecting post-treatment data, the process moves to block 270.

At block 270, the post-treatment data is communicated 290d to catheter-based procedure information module 299 so that such post-treatment data can be read, stored and/or analyzed by catheter-based procedure information module 299, which may be present locally at or near the treatment location, including, in some cases, integrated into the catheter-based system used to apply the catheter-based procedure, or present remotely, e.g., comprising a distributed system available over network connections, e.g., a cloud-based configuration.

Analysis of post-treatment data performed by catheter-based procedure information module 299 may comprise computations or look-ups to reference or historical data for evaluating the effectiveness of the catheter-based procedure (i.e., generating outcome information such as a conclusion about the effectiveness of treatment based on the final data collected from the catheter-based procedure). In some cases, analysis of post-treatment data performed by catheter-based procedure information module 299 comprises comparing the instant post-treatment data with historical post-treatment data or applying a machine learning algorithm to evaluate post-treatment data, such as to the evaluate the effectiveness of the catheter-based procedure based on aspects of the post-treatment data. Upon analyzing post-treatment data by catheter-based procedure information module 299, results of such analysis of post-treatment data by catheter-based procedure information module 299 are communicated 290d to the catheter-based system, e.g., for display and further analysis by an operator or a treatment provider. The process set forth in exemplary flow diagram 200 ends at block 270.

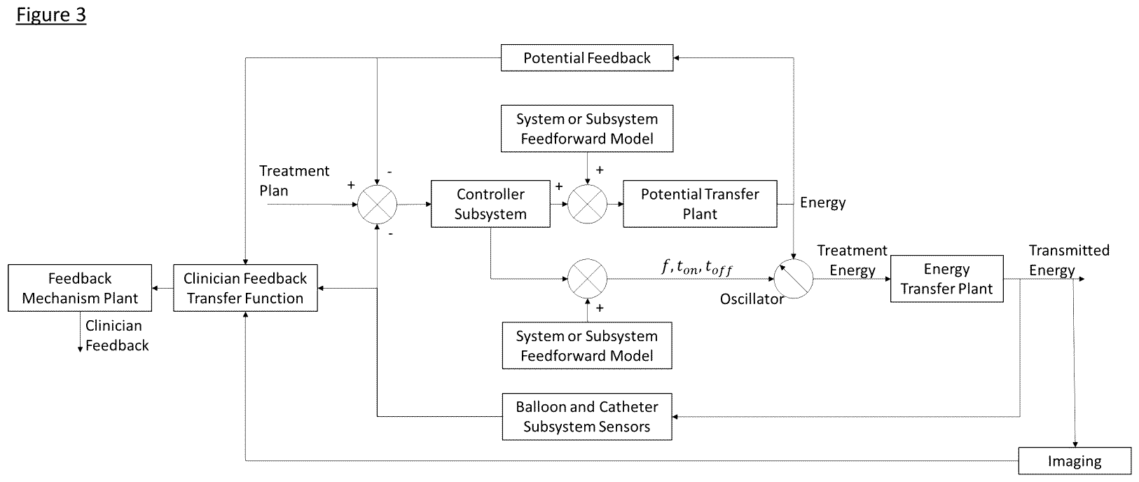

FIG. 3 depicts a control loop schematic for controlling a catheter-based system while performing a catheter-based procedure, e.g., a catheter-based procedure for treating diseased cardiovascular tissue, such as a diseased valve, using, for illustrative purposes, a catheter-based system such as system 100 depicted in FIG. 1. In this embodiment, the controller comprises receiving a treatment plan (i.e., a collection of configurations for the catheter-based procedure, including configurations of the catheter-based system) that is fed into the controller subsystem. The treatment plan may be an initial treatment plan (i.e., a collection of configuration settings for the catheter-based procedure specified prior to initiating the catheter-based procedure) or a revised treatment plan (i.e., a collection of configuration settings for the catheter-based procedure specified sometime after initiating the catheter-based procedure, e.g., a revised treatment plan updated based on peri-treatment data collected while performing the catheter-based procedure), in each case as generated by catheter-based procedure information module, such as catheter-based procedure information module 299 depicted in the embodiment shown in FIG. 2 or catheter-based procedure information module 199 depicted communicating with catheter-based system 100 of FIG. 1.

In the controller embodiment shown in FIG. 3, based at least in part on the treatment plan input to the controller from a catheter-based procedure information module, the controller subsystem sends signals such as in the form of voltage or data to a potential source (regulated or unregulated) to control the amount of potential output by the potential source to an oscillator. The controller subsystem also sends signals to an oscillator, such as frequency (f), i.e., oscillation frequency, and duty cycle (tₒₙ and t_{off}), to control the cyclical energy transmitted by the oscillator. Feedforward models of the individual subsystems (e.g., console assembly, manifold assembly, catheter assembly) or the system as a whole, may be injected into the controller signal (i.e., the controller signal may be augmented with such data regarding a feedforward model) to improve the convergence of the controller (i.e., a control algorithm) to the correctly supplied energy.

Therapy energy (i.e., potential energy) is transmitted to the oscillator, but sensors may be used to track the magnitude of potential energy transmitted to the oscillator. The sensor signals may be fed back into the controller (and ultimately to the catheter-based procedure information module (not shown) used for generating one or more treatment plans) such that they may be used to converge the output of system (i.e., pulsatile energy applied to cardiovascular tissue) to the desired output. The oscillator is turned on and off at an appropriate frequency and duty cycle such that energy is transmitted in an appropriate fashion to the distal location (i.e., to a heart-tissue-conforming element and ultimately to cardiovascular tissue), accounting for any attenuation, heat transfer, bubble formation, tissue relaxation, and the like. Sensors measure system attributes, as well as characteristics of the tissue, e.g., cardiovascular tissue, such as pressure, volume, temperature, flow and the like. Sensor data from the catheter assembly are fed back into the controller so that such measurements may be used to converge the output of the system (i.e., pulsatile energy applied to cardiovascular tissue) to the desired output, e.g., as specified in a treatment plan input to the controller.

Catheter assembly sensors may be located at any convenient location of the catheter assembly where they can be configured to produce an appropriate measurement that can be used for controlling the system. For instance, the sensors may be pressure or volume sensors located on a connector. In other instances, such sensors may be sensors located in the catheter or heart-tissue-conforming element (e.g., in a balloon of a heart-tissue-conforming element) or angioplasty balloon or the like and are connected to wires configured to pass through the catheter. In other cases, such sensors may comprise an X-ray image, or results of another fluoroscopy imaging technique, of the catheter or heart-tissue-conforming element or angioplasty balloon or the like, which is used to determine position or expansion of one or more balloons of the catheter, heart-tissue-conforming element or angioplasty balloon or the like.

The catheter assembly may comprise one or more sensors configured to measure information at one location as a means for determining conditions elsewhere. For instance, a pressure and flow transducer may be configured to measure the pressure and flow rate at the connector, but, for example, combined with a fluid model of the system and pre-treatment data or peri-treatment data comprising, e.g., X-ray or CT imaging, the sensor data may be used to gauge the pressure and/or volume of, for example, an angioplasty balloon or a balloon of the heart-tissue-conforming element or the like. In addition, sensor data may be provided to a clinician feedback transfer function, where such sensor feedback may be combined with feedback generated from the potential transfer plant or imaging data, for processing to a feedback mechanism plant for providing feedback on system behavior to a provider, i.e., operator of the system.

In other instances, a pressure and flow rate sensor may be used to measure the compliance or change in compliance of an angioplasty balloon or a balloon of the heart-tissue-conforming element or the like. Given that diseased tissue provides a resistance to balloon expansion, the amount of volume forced into the balloon at a certain pressure is an indirect indication of the compliance of the tissue surrounding the balloon. In this way, a measure of compliance or change in compliance may be used as a treatment metric or goal, and such may be specified in the treatment plan input to the controller.

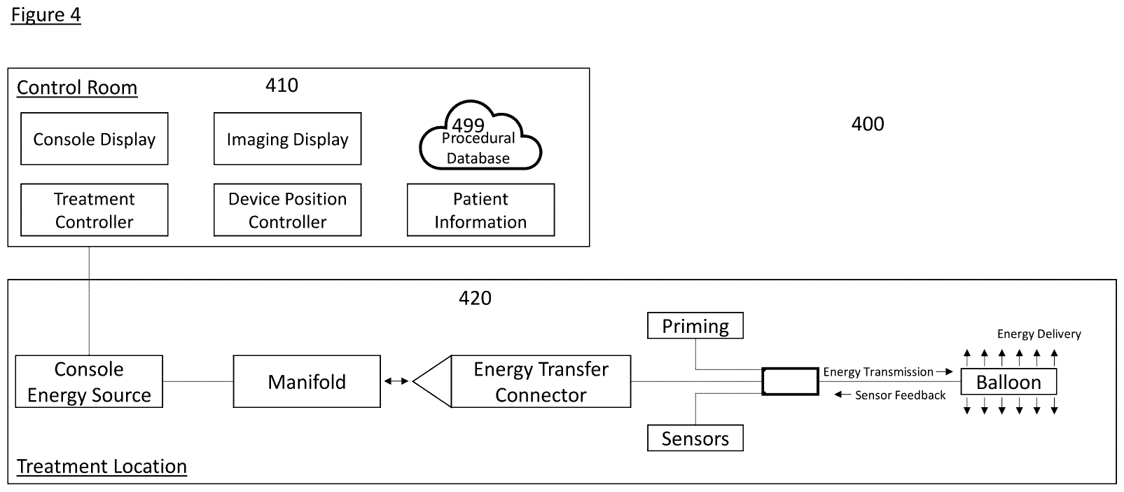

FIG. 4 depicts a schematic of a system configuration 400 for use with an embodiment of a method of the present invention. System configuration 400 may be configured to provide a catheter-based procedure using a catheter-based system, such as system 100 depicted in FIG. 1, and, further, may be used in connection with a robotic technique for delivering therapy energy to diseased tissue, such as a diseased valve, operated from a control room. In configuration 400 the catheter-based system is partitioned into physically separate control room 410 and treatment location 420, which may be separated, e.g., in different partitions of a room or in the next room or in different cities or states or countries.

Control room 410 may comprise a console display, an imaging display, a treatment controller, a device position controller, patient information as well as a connection to procedural database 499, which is a catheter-based procedure information module, present on, or accessed remotely via a computer network connected to, a computer system present in control room 410. Console displays may be used to provide important information about the state of the catheter-based procedure and the catheter-based system relevant to an ongoing treatment, such as, for example, pressure or volume measurements or other information about, for example, a heart-tissue-conforming element, such as the type of heart-tissue-conforming element employed. Information provided on the console displays may be updated periodically during treatment, e.g., substantially in real time. Imaging displays may be used to provide imaging information, such as fluoroscopy imaging results, e.g., showing the position of the heart-tissue-conforming element relative to cardiovascular tissue, e.g., a heart valve. Fluoroscopy imaging results may also provide information about the state of aspects of the heart-tissue-conforming element, such as, e.g., whether a balloon is inflated and to what extent it is inflated. Treatment controller may be used to adjust an aspect of how the catheter-based system is applied to provide treatment, e.g., apply pulsatile energy to the cardiovascular tissue, such as, for example, adjusting a pressure or frequency or duty cycle setting. In embodiments, a treatment controller may adjust configurations of the catheter-based system based at least in part on a treatment plan, such as, for example, an initial treatment plan generated by procedural database 499 (in turn based on pre-treatment data collected, e.g., in treatment room 420 and transmitted to control room 410 for communication to and/or from procedural database 499) or an intermediate or a revised treatment plan generated by procedural database 499 (in turn based on pre-treatment data and/or peri-treatment data collected, e.g., in treatment room 420 and transmitted to control room 410 for communication to and/or from procedural database 499).

Device position controller may be used to adjust the position of the device, i.e., one or more aspects of the catheter-based system, relative to cardiovascular tissue undergoing treatment, e.g., make adjustments to a catheter position to move an angioplasty balloon or a heart-tissue-conforming element or the like more proximally or distally. Patient information may comprise a display of any important patient data relevant to a treatment, such as, for example, information about a treatment site, a patient age, disease state, vitals measurements, such as blood pressure or pulse oximeter readings, for review by a clinician or operator.

Procedural database 499 (i.e., catheter-based procedure information module) comprises relevant information regarding past treatment plans (i.e., information related to catheter-based system configurations for treating cardiovascular tissue, such as oscillation frequencies, duty cycles and/or amplitudes and corresponding details regarding the underlying cardiovascular tissue, such as a degree of calcification), which may be accessed to identify a potential treatment plan in connection with a new treatment. Procedural database 499 may be configured to generate a treatment plan using any convenient technique, i.e., treatment plan algorithm, such as, for example, a look-up-based algorithm that employs matching aspects of a current procedure against past procedures recorded in procedural database 499 or a model-based approach for estimating an optimal treatment plan comprising configurations of the catheter-based system, such as, for example, a statistical model or a machine learning model, including, for example, a neural network model or a deep learning model including, for example, a convolutional neural network model. In embodiments, such a treatment generation algorithm that comprises a machine learning algorithm may be trained using past data and results of catheter-based procedures. In some cases, such a treatment generation algorithm may be configured to be continuously trained based on data collected and results from catheter-based procedures as such procedures on a rolling bases, as such procedures are performed. Procedural database 499 (i.e., catheter-based procedure information module) may be implemented on a computer system present in control room 410 or may be implemented on a computer system present at a remote location and accessed via a computer system present in control room 410.

Treatment location 420 may comprise a system such as catheter-based system 100 depicted in FIG. 1, i.e., an embodiment of a system comprising a console assembly with a potential source, a manifold assembly and a catheter assembly with a connector, a connector-to-catheter transition hub, a catheter and a heart-tissue-conforming element. Treatment room 420 may be, for example, a procedure room or operating room of a hospital or treatment center, for example. Any convenient operable connection capable of transmitting data and control signals between treatment room 420 and control room 410 may be employed, such as a wired or wireless connection, such as via the internet or via a private, dedicated network.

### Measurements of Compliance:

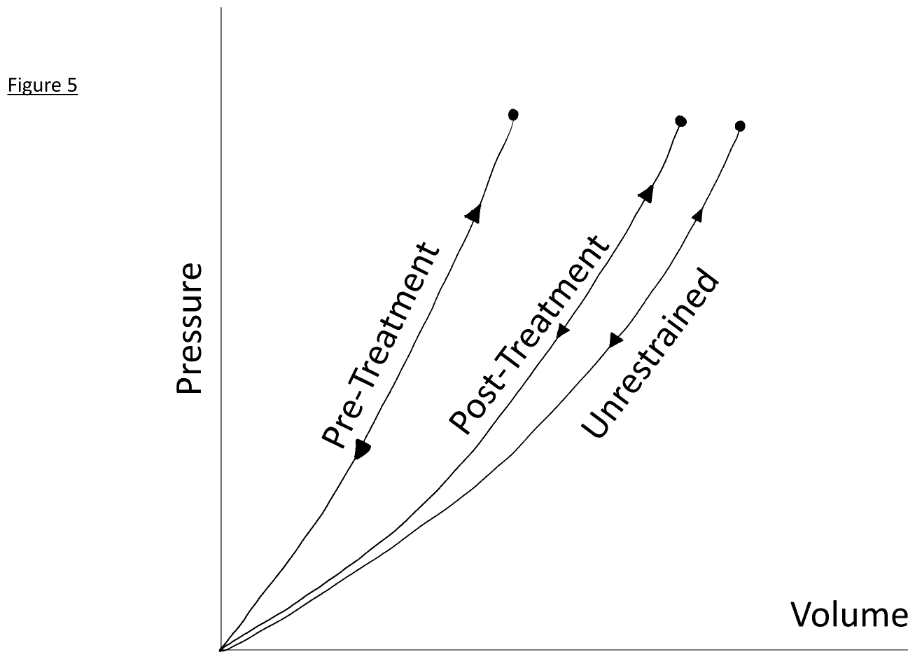

As described in detail below, compliance of a tissue, e.g., cardiovascular tissue or a vessel, is a measurable characteristic of tissue, such as blood vessels or cardiovascular tissue, such as cardiovascular tissue comprising a heart valve, calculated based on a ratio of the change in tissue volume for a given change in pressure. Vessel compliance is an important characteristic for observation because improving vessel compliance is a prerequisite to definitive treatment of certain underlying disease conditions, such as atherosclerosis or the presence of calcifications in tissues such as cardiovascular tissue. Changes in vessel compliance are seen in the different pressure-volume curves depicted in FIG. 5. In FIG. 5, volume is plotted on the x-axis and pressure is plotted on the y-axis. The pressure-volume, i.e., vessel compliance, characteristic of an unrestrained balloon (i.e., a balloon that is not present in tissue or otherwise restrained from expanding its volume upon increase in balloon pressure) versus untreated (i.e., pre-treatment) tissue, i.e., an untreated vessel, versus treated (i.e., post-treatment) tissue, e.g., a treated vessel.

Shown in FIG. 5, upon treatment, e.g., application of pulsatile energy to cardiovascular tissue using an embodiment of a catheter-based system, such as system 100 depicted in FIG. 1, the pressure-volume curve shifts to the right, closer to the curve of the unrestrained balloon. That is, upon treatment, an identical change in tissue volume corresponds to a reduced tissue pressure; i.e., it takes less pressure on the tissue to expand tissue volume a similar amount.

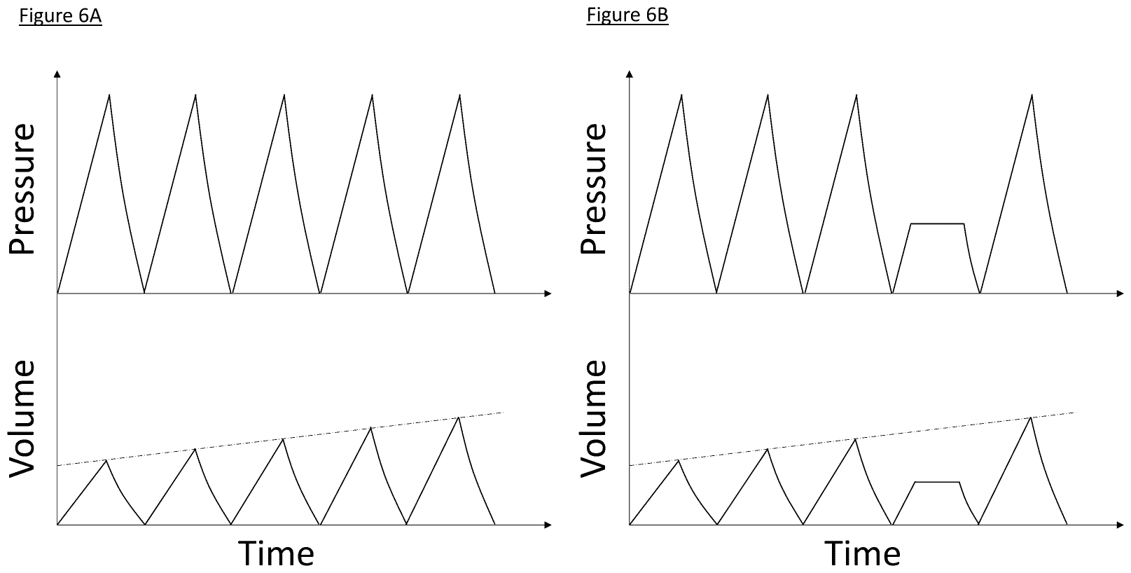

Methods and systems according to the present invention may be configured to assess tissue, e.g., vessel, compliance by obtaining measurements in-vivo of changes in volume at different pressures (or different changes in pressure) applied to cardiovascular tissue such as vessels. FIGS. 6A and 6B provide an example of measurements of changes in tissue compliance obtained during treatment comprising a catheter-based procedure using a catheter-based system, such as, e.g., system 100 depicted in FIG. 1, i.e., during pulsatile lithotripsy within a heart valve. FIG. 6A demonstrates obtaining measurements of tissue compliance, i.e., pressure applied to tissue and corresponding changes in volume over time, using dynamic changes in pressure. FIG. 6A shows peak volume changes trend upward while peak applied pressure remains constant. That is, as treatment progresses, tissue volume increases to a greater extent while the same changes in pressure are applied, an indication of improved tissue compliance.

In embodiments of methods according to the present invention, pre-treatment data, peri-treatment data and post-treatment data may comprise compliance measurements (or data that, together, may be processed to generate compliance measurements) of tissue. In embodiments, information communicated to and/or from the catheter-based information module may comprise tissue compliance data. In such embodiments, the catheter-based procedure information module may comprise a treatment plan generation algorithm that generates a treatment plan, e.g., a revised treatment plan, generated based on peri-treatment data, based on compliance measurements obtained during application of a catheter-based procedure or changes in compliance measurements or based on target compliance goals or target changes in compliance. In some cases, catheter-based procedure information module may be configured to communicate a stopping or ending instruction such that the catheter-based procedure is stopped upon the relevant tissue achieving a specified compliance value or a specified change in compliance. For example, a treatment plan may comprise instructions to cycle pressure applied to a balloon as shown in FIG. 6A with further instructions that should the corresponding balloon volume behave as depicted in FIG. 6A, then such behavior is indicative of desired changes to tissue compliance and the catheter-based procedure should be ended.

FIG. 6B demonstrates obtaining measurements of tissue compliance, i.e., pressure applied to tissue and corresponding changes in volume over time, using dynamic changes in pressure as well as static pressure (i.e., on the third pressure oscillation the pressure applied to tissue remains for approximately half the cycle). FIG. 6B shows peak volume changes trend upward while peak applied pressure remains constant. That is, as treatment progresses, tissue volume increases to a greater extent while the same changes in pressure are applied, an indication of improved tissue compliance.

Embodiments of methods of the present invention may comprise communicating measurements of relative compliance change of cardiovascular tissue (e.g., a vessel) in real time during application of a catheter-based procedure to provide, e.g., pulsatile energy to cardiovascular tissue. Systems of interest for use in performing a catheter-based procedure of the present invention may be configured to measure, and update, treatment parameters based on compliance change of the cardiovascular tissue. For instance, after calcium cracking (i.e., breaking up of calcified plaque tissue), the cardiovascular tissue and angioplasty balloon or heart-tissue-conforming element, or a balloon thereof, or the like may expand significantly, contributing to a large gain in compliance, as measured by the system. However, after the vessel has fully expanded, changes in compliance, as measured by the system, may subside. Identification of such conditions (i.e., different degrees of changes in compliance), as defined in a treatment plan or as identified by a catheter-based procedure information module, may indicate that treatment may be halted because no further appreciable gain is occurring.

Catheter-based systems for use in catheter-based procedures of the present invention may be configured to measure pressure in any convenient manner. In some instances, such systems may comprise a pressure gauge as described herein for measuring pressure in, for example, fluid passages and/or an angioplasty balloon or a balloon of the heart-tissue-conforming element or a catheter or the like. In some instances, a pressure gauge may be installed such that it is configured to measure pressures present in a connector, such as connector 151 in FIG. 1.

Catheter-based systems for use in catheter-based procedures of the present invention may be configured to measure changes in volume of cardiovascular tissue, e.g., a vessel, in any convenient manner. In some instances, embodiments of systems according to the present invention may be configured such that changes in the position of a membrane separating, for example, proximal and distal chambers of a connector (such as connector 151 in FIG. 1) reflect changes in volume of an aspect, e.g., a balloon, of a heart-tissue-conforming element. Changes in the volume of a balloon reflect changes in the cross-sectional area of cardiovascular tissue, such as a vessel, and therefore changes in volume of the cardiovascular tissue. Such catheter-based systems may further comprise a Hall sensor and a permanent magnet for measuring changes in the position of such a membrane. A Hall sensor refers to a sensor configured to sense the presence of, or changes in, a magnetic field, i.e., by use of the Hall Effect. A permanent magnet may be comprised of any convenient magnetic material, or an electromagnet, as desired, such that relative changes in position of the Hall sensor with respect to the permanent magnet are detected by the Hall sensor. Sensors such as the Hall sensor and permanent magnet described above may be used to measure changes in volume of an angioplasty balloon or a heart-tissue-conforming element or the like of a catheter-based system for performing a catheter-based procedure in connection with an embodiment of the present invention, or one or more balloons thereof, as well as the rate at which aspects of such an angioplasty balloon or heart-tissue-conforming element, e.g., one or more balloons thereof, inflates, i.e., the rate of cardiovascular tissue volume change and corresponding changes in compliance.

### Systems

As summarized above, aspects of the present disclosure include systems configured for practicing the subject methods. Systems according to certain embodiments comprise a catheter-based procedure information module, comprising: a first processor comprising memory operably coupled to the first processor, wherein the memory comprises instructions stored thereon, which, when executed by the first processor, cause the first processor to: receive data regarding a catheter-based procedure from a catheter-based system, and transmit data regarding a catheter-based procedure to a catheter-based system. Systems according to certain embodiments further comprise a catheter-based system, comprising: a catheter-based device, a second processor comprising memory operably coupled to the second processor, wherein the memory comprises instructions stored thereon, which, when executed by the second processor, cause the second processor to: transmit data regarding the catheter-based procedure to the catheter-based procedure information module, receive data regarding the catheter-based procedure from the catheter-based procedure information module, and configure the catheter-based device based at least in part on data regarding the catheter-based procedure received from the catheter-based procedure information module. Systems according to certain embodiments further comprise an operable connection between the catheter-based procedure information module and the catheter-based system. Any convenient processors and memories may be employed in embodiments of the invention, such as described above. Any convenient operable connection between the catheter-based procedure information module and the catheter-based system may be employed in embodiments of the invention, such as wired or wireless computer network connections, as described above.

### Catheter-Based Procedure Information Module:

In embodiments of systems of the present invention, the catheter-based procedure information module is remote from the catheter-based system. By “remote,” it is meant that the catheter-based procedure information module is distinct from and separated from the catheter-based system, e.g., on different sides of a partition of a room or in different rooms of a building, different buildings, different cities or different countries. In other cases, the catheter-based procedure information module is integrated into the catheter-based system.

In some cases, the catheter-based procedure information module is configured to store data regarding the catheter-based procedure. For example, the catheter-based procedure information module may be configured to store pre-treatment information, peri-treatment information or post-treatment information, i.e., outcome information related to the catheter-based procedure. In other cases, the catheter-based procedure information module is configured to store data regarding the catheter-based procedure in a distributed data storage. By “distributed data storage,” it is meant that data regarding the catheter-based procedure can stored at more than one storage location or node of a computer network where such nodes may comprise separate physical locations, including, for example, in a cloud-based data storage.

Embodiments of systems of the present invention may comprise more than one catheter-based system and are configured so the catheter-based procedure information module interfaces with each of the catheter-based systems. That is, in instances, the catheter-based system is a first catheter-based system, and the catheter-based procedure information module is configured to receive data from, and transmit data to, a plurality of catheter-based systems. In some cases, in such instances, at least two of the plurality of catheter-based systems are located at different locations, such as, for example, different catheter-based procedure treatment locations.

### Treatment Information:

In embodiments of systems according to the present invention, data regarding the catheter-based procedure transmitted to the catheter-based procedure information module comprises pre-treatment data, peri-treatment data and/or post-treatment data, in each case, as described above. In certain embodiments, the catheter-based device comprises a sensor, and the pre-treatment or peri-treatment or post-treatment data comprises catheter-based device sensor data. In some cases, the catheter-based device sensor data comprises a balloon location, a balloon deployment percentage, a valve opening amount, an eccentricity of the valve, a paravalvular leakage, a surrounding pressure on the valve, a proximal pressure, a distal pressure or a position of the valve in a valve annulus or any other measurement relevant to a catheter-based procedure and capable of detection via a sensor integrated into a catheter-based system.

### Treatment Plan:

In embodiments of systems according to the present invention, the first processor memory further comprises instructions that, when executed by the first processor, further cause the first processor to apply a treatment plan generation algorithm. In some embodiments, the treatment plan generation algorithm is configured to generate a treatment plan based at least in part on data regarding the catheter-based procedure received from the catheter-based system. In other embodiments, data transmitted by the catheter-based procedure information module comprises a treatment plan.

As described above, a treatment plan may comprise, for example, a device type, a balloon type, a treatment type, a treatment duration, a treatment intensity or a treatment frequency. In embodiments, the treatment plan generation algorithm is configured to compare at least a subset of data regarding the catheter-based procedure against data from a previous catheter-based procedure. In other embodiments, the treatment plan generation algorithm is configured to access a collection of data regarding a plurality of previous catheter-based procedures. In some cases, the treatment plan generation algorithm comprises a machine learning algorithm component, which may be trained using data from one or more catheter-based procedures, such as catheter-based procedures previously completed on the same or different subjects performed at one or more locations.

With respect to applying the treatment plan, in some embodiments, configuring the catheter-based device based at least in part on data regarding the catheter-based procedure received from the catheter-based procedure information module comprises configuring the catheter-based device based at least in part on the treatment plan. In some cases, configuring the catheter-based device comprises applying an initial configuration for the catheter-based procedure. In other cases, configuring the catheter-based device comprises re-configuring the catheter-based device.

With respect to configuring the catheter-based device, in embodiments, configuring the catheter-based device comprises, for example, selecting a treatment type, a treatment duration, a treatment intensity or a treatment frequency. In some cases, configuring the catheter-based device comprises automatically adjusting a configuration of the catheter-based device. In other cases, configuring the catheter-based device comprises presenting a change to a configuration of the catheter-based device for operator approval. By presenting a change for operator approval, it is meant, for example, displaying a proposed configuration on an output device, such as a monitor or display, such as a touchscreen display, and receiving input indicating approval or amendment (i.e., a change to an aspect of the treatment plan) such as touching one or more keys on a touchscreen display.

### Catheter-Based Procedures:

In embodiments of systems of the present invention, the catheter-based system may comprise any convenient catheter-based device, such as, for example, catheter-based devices for use in connection with interventional treatments of cardiovascular tissue, such as, for example, balloon catheter devices, and such may vary as desired. In some embodiments, the catheter-based device comprises a pulsatile balloon catheter system. Further details regarding such catheter-based devices which may be employed in connection with the methods systems described herein are provided in U.S. Application No. 63/274,832, the disclosure of which is incorporated herein by reference. In other embodiments, the catheter-based device comprises a microcatheter system for crossing total occlusions. Further details regarding such catheter-based procedures which may be employed in connection with the methods and systems described herein are provided in U.S. Application No. 63/238,381, the disclosure of which is incorporated herein by reference. In still other embodiments, the catheter-based device comprises a system for imparting pulsatile energy to heart valve tissue. Further details regarding such catheter-based procedures which may be employed in connection with the methods and systems are described in U.S. Application Ser. No. 63/346,703 titled “Systems and Methods for Treating Cardiovascular Tissue” and filed on event date herewith (Attorney Docket No. AVSI-004PRV); the disclosure of which is incorporated herein by reference.

### Computer-Readable Storage Media

Aspects of the present disclosure further include non-transitory computer readable storage media having instructions for practicing the subject methods. Computer readable storage media may be employed on one or more computers for complete automation or partial automation of a system for practicing methods described herein. In certain embodiments, instructions in accordance with the methods described herein can be coded onto a computer-readable medium in the form of “programming,” where the term “computer readable medium” as used herein refers to any non-transitory storage medium that participates in providing instructions and data to a computer for execution and processing. Any suitable non-transitory storage medium may be used, such as a floppy disk, hard disk, optical disk, magneto-optical disk, CD-ROM, CD-R magnetic tape, non-volatile memory card, ROM, DVD-ROM, Blue-ray disk, solid state disk, and network attached storage (NAS), whether or not such devices are internal or external to a computer. A file containing information can be “stored” on a computer readable medium, where “storing” means recording information such that it is accessible and retrievable at a later date by a computer.

The following example(s) is/are offered by way of illustration and not by way of limitation.

### EXAMPLES

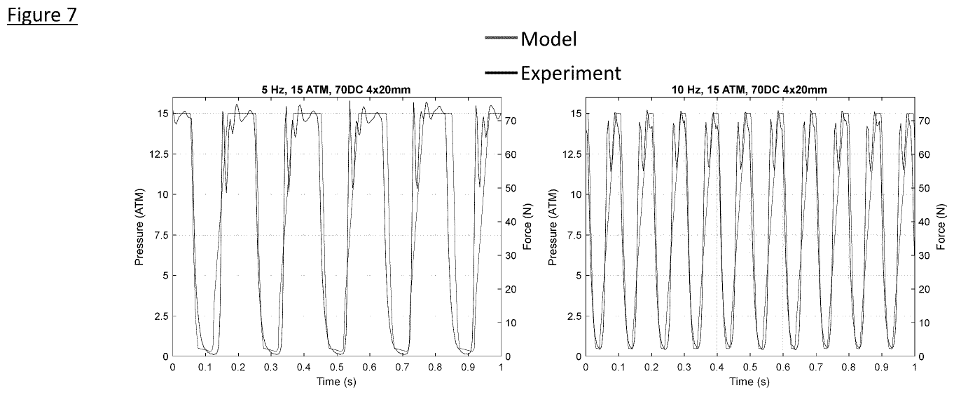

Using measured system characteristics in conjunction with a model of a catheter-based system, embodiments of the present invention may be utilized to control a catheter-based procedure in real time (e.g., using negative feedback control and/or feedforward control) to ensure pressure amplitude, duty cycle, and frequency are appropriately set during such catheter-based procedure. Two examples of experimentally measured pressure in a balloon of a catheter-based system used to perform a catheter-based procedure as well as force output from such balloon are shown in FIG. 7. A system model may be used to predict the experimental measurements accurately. With such a predictive model and with feedback from system measurements (e.g., peri-treatment data, such as sensor data, as described above), procedural characteristics (frequency, pressure input, and duty cycle) can be adjusted to ensure maximized treatment effect. In addition to utilizing such features of embodiments of the present invention to facilitate more effective catheter-based procedures by, e.g., optimizing changes in balloon pressure, such features may also be utilized to improve the safety of catheter-based procedures. For example, a significant change in pressure and/or volume of a balloon that is detected by sensors in or associated with the catheter-based system may cause a catheter-based information module or another controller that is an embodiment of the present invention to automatically shut the catheter-based system down in order to prevent a safety hazard.

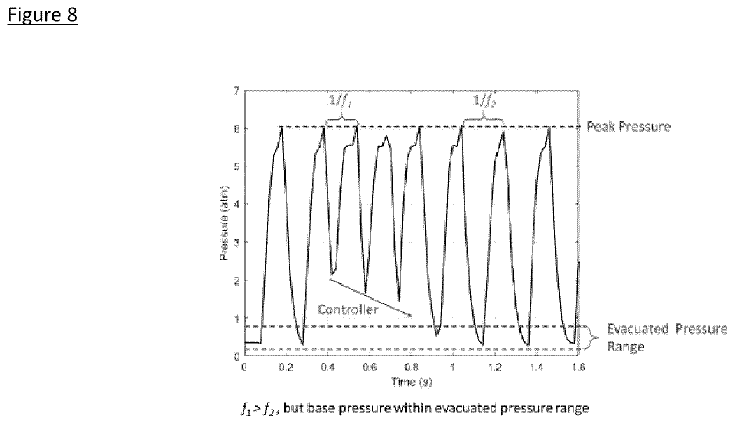

An example application of controlling a catheter-based procedure is shown in FIG. 8. During a catheter-based procedure where a balloon of a catheter-based system is periodically pressurized over time, the balloon depressurization pressure falls out of the desired range. When this occurs, a catheter-based information module (i.e., a control algorithm of a catheter-based system for controlling a catheter-based procedure) works to reduce the frequency of oscillation (i.e., periodic balloon pressurizations) such that the balloon has enough time to depressurize and reach the appropriate pressure range.

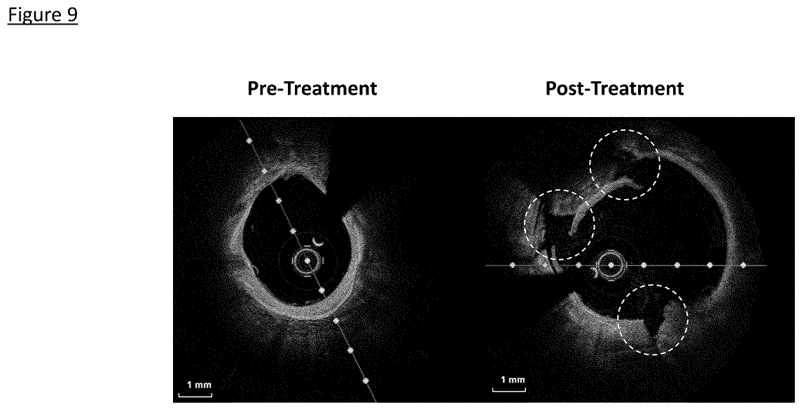

FIG. 9 presents exemplary images of calcifications in luminal tissue prior to and subsequent to applying a catheter-based procedure (pre-treatment and post-treatment). The effects of applying an optimal treatment plan for controlling the catheter-based procedure are seen in the post-treatment image, where the calcification has been disrupted in several locations as a result of applying a catheter-based procedure according to an embodiment of the present invention.

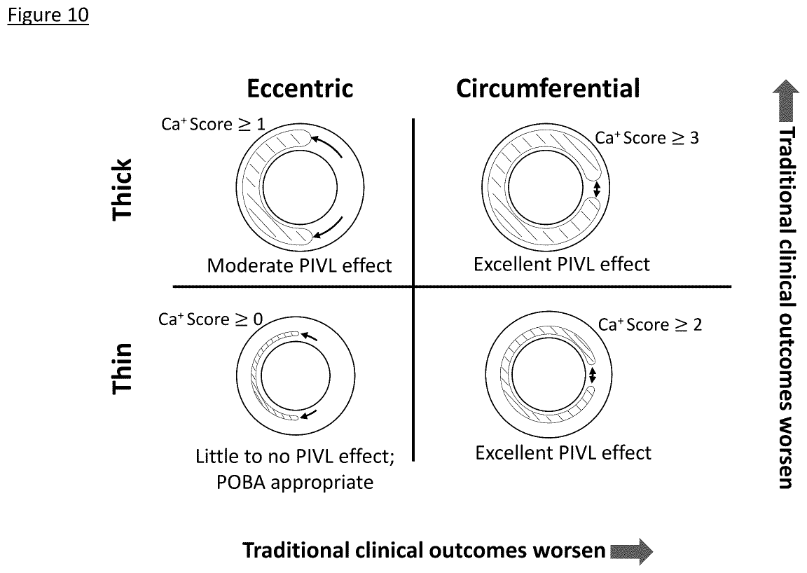

FIG. 10 shows a table summarizing the effectiveness of peripheral intravenous line (PIVL)-based catheter-based procedure approaches under different conditions. The four quadrants of the table present a metric based on different circumstances of when to deliver treatment, i.e., catheter-based procedures, and when such treatment is likely to be most effective. Embodiments of catheter-based information modules, when generating treatment plans, may be configured to apply such metrics. In some cases, thickness of a crack in a calcium deposit may be treated as a treatment objective and treatment plans generated by a catheter-based information module may be based at least in part on such objective. Crack thickness can, for example, be a function of treatment time and pulse energy applied via a catheter-based procedure.

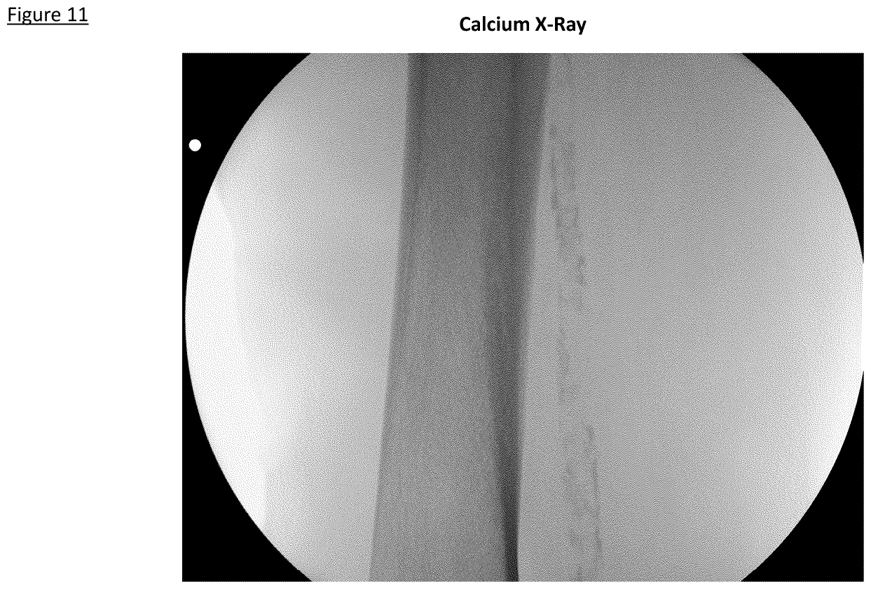

FIG. 11 depicts an exemplary X-ray image showing a calcium deposit in tissue. Such X-ray imaging may comprise information communicated to and/or from a catheter-based information module. That is, such an image, or an annotated version thereof, may comprise pre-, peri- or post-treatment data as described herein. Images, such as the X-ray image shown in FIG. 11 provide information about how dense a calcium deposit is such that a catheter-based information module may respond accordingly in a generated treatment plan; i.e., calcium density or hardness of calcium deposits may be indicated in X-ray images, such as that seen in FIG. 11 based on the greyscale output of the X-ray. Based on one or more images such as that shown in FIG. 11, a catheter-based information module may determine, i.e., modify or optimize, the energy to be applied to such a calcium deposit. For example, a catheter-based information module may generate a treatment plan for applying lower pressure in a catheter-based procedure based on images depicting smaller diameter calcium deposits. Similarly, ultrasound images may be used to measure vascular strain and such strain measurements may be applied by a catheter-based information module to help configure, for example, a magnitude of force applied during a catheter-based procedure or some other treatment configuration. In some cases, intravascular ultrasound images may be used to determine differences in strain across different locations within vessels. Such differences in characteristics of different locations of vessels may be applied by a catheter-based information module to help configure, for example, a magnitude of force applied during a catheter-based procedure or some other treatment configuration.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method comprising:
performing a catheter-based procedure; and
communicating data regarding the catheter-based procedure to and/or from a catheter-based procedure information module.

2. The method according to claim 1, wherein the catheter-based procedure information module is a remote module.

3. The method according to claim 1, wherein the catheter-based procedure information module is configured to store data regarding the catheter-based procedure.

4. The method according to claim 3, wherein the catheter-based procedure information module comprises distributed data storage.

5. The method according to claim 3, wherein the catheter-based procedure information module comprises cloud-based data storage.

6. The method according to claim 1, wherein the catheter-based procedure information module is configured to receive and/or transmit data regarding a plurality of catheter-based procedures.

7. The method according to claim 6, wherein at least two of the plurality of catheter-based procedures are performed at different locations.

8. The method according to claim 7, wherein the different locations comprise different catheter-based procedure treatment locations.

9. The method according to claim 1, wherein data regarding the catheter-based procedure communicated to the catheter-based procedure information module comprises pre-treatment data.

10. The method according to claim 9, wherein the pre-treatment data comprises data collected prior to performing the catheter-based procedure.

11-16. (canceled)

17. The method according to claim 1, wherein data regarding the catheter-based procedure communicated to the catheter-based procedure information module comprises peri-treatment data.

18. The method according to claim 17, wherein the peri-treatment data comprises data collected during the catheter-based procedure.

19-26. (canceled)

27. The method according to claim 1, wherein data regarding the catheter-based procedure communicated to the catheter-based procedure information module comprises post-treatment data.

28-38. (canceled)

39. The method according to claim 1, further comprising configuring the catheter-based procedure based at least in part on data communicated from the catheter-based procedure information module.

40. The method according to claim 1, wherein the catheter-based procedure information module is configured to implement a treatment plan generation algorithm.

41-58. (canceled)

59. The method according to claim 1, wherein the catheter-based procedure comprises a method of imparting pulsatile energy to an internal luminal tissue location.

60-64. (canceled)

65. The method according to claim 1, wherein the catheter-based procedure is performed on a subject.

66. The method according to claim 65, wherein the subject is mammalian.

67. The method according to claim 66, wherein the subject is human.

68. A system comprising:
a catheter-based procedure information module, comprising:
a first processor comprising memory operably coupled to the first processor, wherein the memory comprises instructions stored thereon, which, when executed by the first processor, cause the first processor to:
receive data regarding a catheter-based procedure from a catheter-based system; and
transmit data regarding a catheter-based procedure to a catheter-based system;
a catheter-based system, comprising:
a catheter-based device;
a second processor comprising memory operably coupled to the second processor, wherein the memory comprises instructions stored thereon, which, when executed by the second processor, cause the second processor to:
transmit data regarding the catheter-based procedure to the catheter-based procedure information module;
receive data regarding the catheter-based procedure from the catheter-based procedure information module; and
configure the catheter-based device based at least in part on data regarding the catheter-based procedure received from the catheter-based procedure information module; and
an operable connection between the catheter-based procedure information module and the catheter-based system.

69-170. (canceled)
